(19) United States
(12) Patent Application Publication
(10) Pub. No.: US 2026/0096737 A1
(43) Pub. Date: Apr. 9, 2026

(54) AN APPARATUS FOR SENSING A BIOLOGICAL TISSUE

(71) Applicant: Creo Medical Limited, Chepstow (GB)

(72) Inventors: Philip ANTHONY, Chepstow (GB); Christopher Paul HANCOCK, Chepstow (GB); Warren JONES, Chepstow (GB)

(21) Appl. No.: 19/114,363

(22) PCT Filed: Sep. 21, 2023
(86) PCT No.: PCT/EP2023/076102

§ 371 Date: Mar. 21, 2025

(30) Foreign Application Priority Data
Sep. 23, 2022 (GB) 2213953.9

(51) Int. Cl. A61B 5/0507; A61B 5/00; A61B 18/00; A61B 18/14; A61B 18/18

(52) U.S. Cl. CPC A61B 5/0507; A61B 5/4836; A61B 5/4848; A61B 5/6847; A61B 5/6876; A61B 5/7225; A61B 5/746; A61B 18/1482; A61B 18/1815; A61B 2018/00345; A61B 2018/00666; A61B 2018/00708; A61B 2018/00761; A61B 2018/00785; A61B 2018/00875; A61B 2018/00892; A61B 2018/1823

(57) ABSTRACT

Various aspects provide an apparatus for sensing a condition or type of a biological tissue. The apparatus comprises a control circuit. The control circuit comprises an electrical connection for delivering electrical signals to the biological tissue. The control circuit is adapted to: deliver an electrical signal to the biological tissue for an active time period; and obtain a charge measurement of an amount of charge stored in the biological tissue in response to the electrical signal during a sampling period after the active time period. Other related apparatuses and systems are also provided.

### FIELD OF THE INVENTION

The present invention relates to an apparatus for obtaining a measurement from a biological tissue and particularly, although not exclusively, to apparatus for obtaining a measurement from a vessel being sealed. In an embodiment, the measurement is used sense a condition and/or type of the tissue, for example, when the vessel has been sealed.

### BACKGROUND

There has been a significant amount of development in the field of electrosurgical devices for delivering therapeutic treatments to a subject or patient. In particular, there has been a focus on the development of electrosurgical devices for delivering therapeutic treatments to an internal portion of the subject through a small opening or incision.

For example, such devices may include an elongate probe or a catheterised portion for accessing an internal region of the subject that is difficult or dangerous to access through open surgery. In some examples, such devices may include a means of delivering electromagnetic energy or radiation to the treatment site within the subject.

One drawback of such devices is that the area being treated is often out of view of the clinician providing the treatment and operating the device. It is therefore often difficult to judge whether the treatment has been fully completed. This can lead to either incomplete treatment at the treatment site, or an over application of the treatment in order to ensure that the treatment has been completed. However, in the cases of apply electromagnetic energy or radiation to the treatment site, an over application of the electromagnetic energy or radiation can result in unnecessary tissue damage at or around the site being treated.

There have been attempts to overcome this issue by providing cameras along with the treatment probe in order to provide the clinician with a view of the treatment in progress. However, such visual aids are often incapable of providing the clinician with a full understanding of the progress of the treatment. For example, the view of the camera is often obscured by tissue surrounding the treatment site or the treatment itself may not result in a clear visual indication showing when the treatment is completed.

In further attempts to overcome this issue, there have been developments to obtain measurements from the tissue in an attempt to determine the condition of the tissue as it undergoes treatment. Such attempts have been directed to sensing an electronic impedance of the tissue in order to monitor the condition of the tissue. However, not only is electrical impedance a complicated measurement to obtain, but the results also obtained from biological material are often extremely noisy and inaccurate. Further, the measure of impedance is also subject to noise from the capacitance of the probe being used, further affecting the accuracy of the measurements obtained from the tissue.

The present invention has been devised in light of the above considerations.

### SUMMARY OF THE INVENTION

In a first aspect, there is provided an apparatus for sensing a condition and/or type of the biological tissue, the apparatus comprising: a control circuit, wherein the control circuit comprises an electrical connection for delivering electrical signals to the biological tissue, and wherein the control circuit is adapted to: deliver an electrical signal to the biological tissue for an active time period; and obtain a charge measurement of an amount of charge stored in the biological tissue in response to the electrical signal during a sampling period after the active time period.

The invention provides a means of obtaining a measurement of the amount of charge that can be stored in the biological tissue being investigated. The amount of charge that can be stored by a biological tissue may be indicative of a condition and/or type of the biological tissue.

Put another way, there is provided a means of measuring an amount of charge that can be held in a biological material by first imparting a charge to the biological tissue, for example by applying a voltage across the tissue, and then measuring the amount of charge remaining in the tissue after a given amount of time.

In other words, there is provided a means of obtaining a direct electrical measurement indicative of tissue condition. In an embodiment, a “condition” is taken to mean a state of the tissue undergoing treatment and, in particular, a state indicative that the treatment is complete. For example, where the tissue is a vessel, such as a blood vessel, and the treatment is to seal the vessel, then the condition is that the vessel has been sealed. In another example, where the tissue is a tumour, such as a cancerous tumour, and the treatment is to ablate or desiccate the tumour, then the condition is that the tumour has been ablated or desiccated. In a further example, the condition of the biological tissue may be considered to be the current cellular structure of the biological tissue being treated. For example, the condition of the biological tissue may comprise a water content of the biological tissue.

In addition to being indicative of the condition of the tissue, the measurement may also be indicative of tissue type. Different tissue types to be differentiated by obtaining a charge measurement across a range of frequencies. Thus, the charge measurement may be indicative of tissue type and/or condition if performed over a range of frequencies. Information about the tissue condition and type may be extracted by looking at the relative amplitude of the charge measurement signals at different frequencies in a manner similar to dielectric spectroscopy. The tissue type may include one or more of: an epithelial tissue; a connective tissue; a muscle tissue; a nervous tissue; a fat tissue; an organ tissue; a lung tissue; a heart tissue; a liver tissue; a pancreatic tissue; a gastric tissue; a renal tissue; a kidney tissue; and a vessel. The tissue type, and boundaries between tissue types, may be derived from the measurements in order to adjust the control of the electrical signals based on the tissue type in contact with the electrical connection.

For example, when cutting into tissue in the gastrointestinal (GI) tract, it may be difficult to cut through the layers of mucosa, sub-mucosa and epithelium without cutting into the muscle tissue surrounding the GI tract. Accordingly, by deriving tissue type, and in particular changes in tissue type, from the measurements, the application of cutting EM energy can be controlled and/or stopped based on the detected tissue type. Tissue type can be determined by detecting the lowest frequency (i.e., the clocking frequency of the system, which determines the length of the active and sampling periods), at which measurable charge storage occurs, for example muscle can only store charge for a short time relative to the other tissue types (mucosa, sub-mucosa and epithelium). This detection of changes in tissue type may be linked to the alarm and alert systems described herein in order to inform the clinician of what tissue type is in contact with the probe.

Put another way, there may be provided an electrosurgical apparatus for controlling energy delivery to a biological tissue, the apparatus comprising: a control circuit, wherein the control circuit comprises an electrical connection for delivering electrical signals to the biological tissue, and wherein the control circuit is adapted to: deliver an electrical signal to the biological tissue for an active time period; obtain a charge measurement of an amount of charge stored in the biological tissue in response to the electrical signal during a sampling period after the active time period; determine a tissue type of the biological tissue based on the charge measurement; and generate a control signal for delivery to an energy generator for controlling the delivery of energy to the biological tissue based on the determined tissue type.

Delivering the electrical signal may comprise delivering an electrical signal at a plurality of signal frequencies. For example, the electrical signals may be delivered to the biological tissue at two or more different frequencies. In another example, the electrical signal may be swept through a plurality of frequencies, such as through a frequency range of interest. For each of the plurality of frequencies, or at regular intervals across the frequency sweep, charge measurements may be obtained. The amount of charge stored by a tissue is dependent of the frequency of the signal delivering the charge (i.e., the clocking signal of the system switching between active and floating modes). The plurality of charge measurements may therefore provide a frequency response profile of the tissue being measured, which may be used to identify the tissue, for example based on a peak charge measurement from the plurality of charge measurements (i.e., the frequency at which the greatest amount of charge is stored) or a minimum charge measurement from the plurality of charge measurements (i.e., the lowest frequency at which charge storage can be measured, which may be compared to a threshold value).

In the GI tract example outlined above, the system may then differentiate between different tissue types for controlling the energy delivery of the electrosurgical system. For example, if the determined tissue type is a first type (e.g. muscle), the control signal may comprise a stop signal for stopping the delivery of energy to the biological tissue. Whereas, if the tissue type is a second type (e.g. one or more of mucosa, sub-mucosa and epithelium), generating the control signal may comprise generating a delivery signal for starting, or continuing, the delivery of energy to the biological tissue.

The measurement of the charge stored in a tissue is less subject to signal noise, meaning that the measurement obtained is more accurate. The measurement of the charge stored in a tissue is also not subject to the resistance or capacitance of the electrical structure being used to obtain the measurement, and can therefore be obtained using a simpler electronic arrangement without negatively impacting the accuracy of the results.

The present invention provides a more accurate means of determining between different tissue types compared to conventional methods. For example, impedance measurements alone are not sufficient to perform this function accurately. Whilst muscle does have a lower impedance than mucosa, sub-mucosa and epithelium, a small contact area of muscle has a similar impedance to a large contact area of these higher impedance tissues. Accordingly, with impedance measurement cutoff alone, it is therefore still possible to puncture the muscle wall by just making contact with the instrument tip, for example. For similar reasons, using impedance shut-off in a jaw-like instrument such as a vessel sealer is liable to false measurement due to varying jaw closure force or pressure. For example, an increase in impedance which represents a vessel being sealed might be falsely detected due to the jaw pressure being reduced on an unsealed vessel.

Further, unlike impedance measurement, using the charge storage methods of the present invention it is possible to sense tissue condition and type whilst the surgical instrument is contaminated, for example by blood or saline. By contrast, measuring tissue using impedance between electrodes can be ‘shorted out’ by contaminating fluids on the instrument, which is a likely condition during normal use.

The sensing technique of the present invention excels in situations where the contact area between tissue and instrument is unpredictable or variable, and/or the instrument is likely to become immersed in or contaminated by fluids, and/or applications requiring a long cable between the generator and the instrument such as robotic or endoscopic applications.

The electrical signal may be any electrical signal for imparting a charge to the biological tissue. For example, the electrical signal may be a DC voltage. The sampling period may be the same length as the active time period or it may be a different length to active time period. The sampling period may occur immediately after the active time period or there may be a delay between the active time period and the active time period. The electrical connection may be a direct connection between the control circuit and the biological tissue or an indirect connection between the control circuit and the biological tissue.

The control circuit may comprise a switching unit adapted to switch the control circuit between a plurality of operation modes. The plurality of operation modes may comprise an active mode, wherein the electrical connection is connected to an electrical signal source during the active time period and a floating mode, wherein the electrical connection is connected to a sensing ground during the sampling period, thereby obtaining the charge measurement. The switching unit is adapted to switch the control circuit to the active mode during the active time period and to the floating mode during the sampling period. The sensing ground may comprise any circuity suitable for obtaining the charge measurement, such as a sensing ground, such as a terminal of a capacitor.

In this way, the apparatus may switch between a number of different functions in order to both impart a charge to the biological tissue in question and measure the charge stored by said tissue. By connecting the electrical connection to a sensing ground, the measurement of the charge stored by the biological tissue may be performed using the same circuitry that initially provided the charge to the tissue.

The active mode may comprise a first active mode, wherein the electrical connection is connected to an electrical signal source having a first polarity and a second active mode, wherein the electrical connection is connected to an electrical signal source having a second polarity, opposite the first polarity. The switching unit may be adapted to select one of the first active mode and the second active mode during the active mode. The switching unit may be adapted to alternate between selecting the first active mode and selecting the second active mode between subsequent active modes. In this way, the tissue is not subjected to a net DC charge thereby improving the safety of the apparatus. The first polarity may be positive and the second polarity may be negative, or vice versa.

The switching unit may be any switch for changing the operation of the control circuit. In particular, the switching unit may be any switch for changing the connection of the electrical connection between the electrical signal source and the sensing ground. For example, the switching unit may comprise one or more switches, such as a switching array. In a further example, the switching unit may comprise a multiplexer adapted to selectively connect the electrical connection to the electrical signal source or the sensing ground.

The electrical signal source may provide any electrical signal. For example, the electrical signal source may provide a positive or negative DC voltage. In a particular example the electrical signal source may provide a voltage of ±5V. The sensing ground may comprise any suitable floating connector for receiving and obtaining the measurement. The sensing ground may include a sampling unit for receiving the measurement. For example, the sampling unit may comprise a capacitor, such as a 1 nF capacitor.

The plurality of operation modes may further comprise a reference ground mode. The electrical connection may be connected to a reference ground during the reference ground mode. The switching unit may be adapted to switch the control circuit to the reference ground mode after the active time period and before the sampling period. The length of time the electrical connection is connected to the reference ground for may be the same as the active time period and/or the sampling period or it may be different.

By connecting the electrical connection to a reference ground after the active mode, any remaining surface charges may be removed from the biological tissue. Thus, the measurement obtained only represents the charge actually stored within the tissue being investigated and therefore the accuracy of the measurement is improved. In other words, by removing the surface charges from the tissue before obtaining the measure of the amount of charge stored in the biological tissue is obtained, the accuracy of the measure may be improved.

The plurality of operation modes may further comprise an error compensating mode. The electrical connection may be connected to an offset signal source during the error compensating mode. The offset signal source may have a lower amplitude than the signal provided to the biological tissue in the active mode. The offset signal source may be a DC voltage source. In a particular example, the offset signal source may provide a voltage of less than 1V, such as less than or equal to 100 mV, such as 10 mV. The switching unit may be adapted to switch the control circuit to the error compensating mode after the active time period and before the sampling period. In the case where the plurality of operation modes also comprises a reference ground mode as described above, the switching unit may alternate between selecting the reference ground mode and the error compensating mode between subsequent active modes.

For example, a sequence of operation modes may run as active mode, floating mode, repeat. In a further example, the sequence of operation modes may run as first active mode, floating mode, second active mode, floating mode, repeat. In a further example, the sequence of operation modes may run as active mode, reference ground mode, floating mode, repeat. In a further example, the sequence of operation modes may run as first active mode, reference ground mode, floating mode, second active mode, reference ground mode, floating mode, repeat. In a further example, the sequence of operation modes may run as active mode, error compensating mode, floating mode, repeat. In a further example, the sequence of operation modes may run as active mode, error compensating mode, floating mode, active mode, reference ground mode, floating mode, repeat.

In a preferred example, the sequence of operation modes may run as first active mode, error compensating mode, floating mode, second active mode, reference ground mode, floating mode, repeat. However, any sequence of operation modes may be utilized according to the application of the invention. The control circuit may further comprise an amplifier unit adapted to amplify the charge measurement. In this way, small amounts of charge may still be detected by the apparatus. The amplifier unit may be connected to the sensing ground. The amplifier unit may comprise a first amplifier circuit adapted to amplify the charge measurement obtained in the floating mode following the first active mode and a second amplifier circuit adapted to amplify the charge measurement obtained in the floating mode following the second active mode. The amplifier unit may further comprise a third amplifier circuit adapted to obtain a difference value from the first amplifier circuit signal and the second amplifier circuit signal. The amplifier unit may comprise any suitable means of amplifying an electrical signal. For example, the amplifier unit may comprise one or more operational amplifiers.

The control circuit may comprise an input for receiving a control signal. The input may be any suitable electrical input port. The active time period may be based on the control signal. Put other way, the length of the active time period may be determined based on the control signal, for example based on a frequency of the control signal. The switching between operation modes of the control circuit performed by the switching unit may be performed according to the frequency of the control signal. For example, the control signal may be a square wave signal and the switching unit may be adapted to switch the control circuit between the operation modes in response to the rising edge, or the falling edge, of the control signal. The apparatus may comprise a signal generator in communication with the input, which is adapted to generate the control signal. The signal generator may comprise any suitable means for generating a signal, such as a dedicated signal source, a single frequency signal source, a variable frequency signal source, an analogue signal generator, a vector signal generator, and the like. The signal generated by the signal generator may be a square wave signal and may have a frequency between 100 Hz and 100 MHz.

For example, the frequency of the signal generated by the signal generator may be 32 kHz or it may be within the radiofrequency (RF) range, such as 500 kHz. The active time period may be a single period of the control signal, multiple periods of the control signal or a fraction of a period of the control signal. In a preferred embodiment, the active time period may be a half period of the control signal.

The control signal may be swept between a plurality of frequencies. For example, the frequency of the control signal may be altered after each sequence of operational modes has completed. By way of a specific example, the control circuit may be cycled through a first active mode, an error compensating mode, a floating mode, a second active mode, a reference ground mode and a floating mode using a control signal at 500 kHz in a first sequence. The control circuit may then be cycled through the same sequence of operational modes using a control signal at 32 kHz in a second sequence. The control signal may then be switched alternately between the two frequencies. In this way, measurements at two, or more, different frequencies may be obtained from the biological tissue in the same measurement operation. By using two different measurements at different frequencies, the dependence of the accuracy of the measurement at a given frequency may be removed and the accuracy of the final measurement improved. The measurement may also be performed at more than two frequencies and each measurement may be used in combination with the other measurement or separately for sensing the condition of the tissue. The relative change of the charge measurement between different frequencies may be used as an indicator of tissue type due to dielectric relaxation effects in different tissues.

The relative change of the charge measurement between different frequencies may also be used as an indicator of tissue condition due to the change in dielectric relaxation effects of a given tissue as it is treated, for example as it is sealed or heated. In an example where the biological tissue is vessel tissue to be sealed, the progress of the sealing of the vessel may be monitored based on a change in the charge measurement at different frequencies. For example, when unsealed the charge measurement obtained from the vessel would be lower at a lower frequency and higher at a higher frequency, whereas when the vessel is sealed, the charge measurement obtained from the vessel would be higher at the lower frequency and lower at the higher frequency. Known frequency responses for vessel tissue may be used to select the frequencies used to monitor vessel sealing, and may include two or more frequencies for monitoring.

The control circuit may further comprise a counter adapted to generate a clocking signal based on the control signal. The switching unit may be adapted to switch the control circuit between the plurality of operation modes based on the clocking signal. For example, the switching unit may be adapted to cycle the control circuit through the plurality of operation modes based on the clocking signal. In this way, the control circuit may be automatically switched between operation modes according to a desired timing scheme. Further, multiple measures of the amount of charge stored in the biological tissue may be taken over time. Thus, it may be possible to monitor the condition of a tissue, and how it changes, over time, for example in response to treatment. The clocking signal may comprise one or more of: the control signal, wherein the control signal has a control signal period; a first clock signal having a first clock signal period, the first clock signal period being different to the control signal period; and a second clock signal having a second clock signal period, the second clock signal period being different to the control signal period and the first clock signal period.

According to a further aspect, there is provided an electrosurgical system for treating a biological tissue, the electrosurgical system comprising: an apparatus as described above; and a probe having a distal tip portion for delivering the electrical signal into the biological tissue and for obtaining the charge measurement from the tissue, wherein the probe is in electrical communication with the electrical connection. The electrosurgical system may be any system adapted to utilize an electrical signal in the treatment of a tissue. The distal tip portion of the probe may comprise a pair of electrodes, the distal tip portion for contacting the biological tissue and obtaining the measurement. The pair of electrodes may act as an active electrode and a return electrode for the electrical signal being provided to the biological tissue and for obtaining the measurement from the biological tissue. The probe may comprise an elongate portion at one end of which is provided the distal tip portion. In an embodiment, the probe is configured to deliver radiofrequency electromagnetic (EM) energy and/or microwave EM radiation separately or simultaneously from a distal end thereof. In an embodiment, a suitable probe may be as described in WO2011010086A1. In addition, the probe may be provided with separated sensing electrodes and cutting electrodes. Put another way, the probe may comprise a first set of electrodes for performing the sensing and tissue treatment functions, such as vessel or tissue sealing, and a second set of electrodes specifically for cutting the tissue using RF EM energy.

The electrosurgical system may further comprise a processor in communication with the apparatus. The processor may be adapted to: receive the charge measurement from the control circuit; and determine a condition and/or type of the biological tissue based on the received charge measurement. For example, the processor may be adapted to determine if the tissue has been treated based on the charge measurement. The processor may be adapted to determine the condition and/or type of the biological tissue based on the measurement using a look-up table, a user defined condition, a neural network, and the like. The biological tissue may be a vessel and the treatment being applied may be sealing the vessel, in which case the processor may be adapted to determine when the vessel has been sealed based on the measurement (e.g., the condition may be sealing of the vessel). For example, a change in the measurement of the charge stored by the tissue may be used to determine that the vessel has been sealed. In a further example, the measurement of the charge stored by the tissue being greater than or less than a predetermined threshold may be used to determine that the vessel has been sealed. Further, a rate of change of the measurement crossing (e.g., exceeding) a predetermined value may also be used to determine that the vessel has been sealed. Further, the rate of change of the measurement approaching a steady state may also be used to determine that the vessel has been sealed.

The biological tissue may also be any other type of tissue undergoing treatment, and in particular undergoing treatment involving the delivery of energy to the biological tissue. The delivery of energy to a biological tissue causes the structure of the biological tissue to change, which in turn changes the amount of charge that can be stored in the biological tissue. For example, the biological tissue may be a tissue being resected, desiccated, ablated and the like. For example, the biological tissue may be a tumour undergoing ablation. In such cases, the condition being sensed may be: tissue resection, tissue desiccation, tissue ablation, respectively.

The electrosurgical system may further comprise a microwave signal generator for generating microwave electromagnetic (EM) radiation. In this case, the probe may be further adapted to deliver the microwave EM radiation. In particular, the probe may be adapted to emit the microwave EM radiation from the distal tip portion of the probe. In this way, the system may deliver microwave EM radiation to the tissue and monitor a condition of the tissue, by way of the charge measurement, using the same apparatus. The pair of electrodes provided at the distal tip portion of the probe may act as the radiating antenna for the microwave EM radiation. In an embodiment, the microwave signal generator may be an electrosurgical generator, for example, as disclosed in WO 2012/076844. Since this electrosurgical generator is also capable of delivering RF EM energy, a single electrosurgical generator may be able to provide both the control signal for the apparatus for sensing a condition of the biological tissue, and a treatment signal (e.g., microwave EM energy) for treating the biological tissue. In this way, the control circuit maybe connectable to the electrosurgical generator for receiving the control signal therefrom.

The processor may be further adapted to: activate the apparatus for obtaining the charge measurement from the biological tissue and deactivate the microwave signal generator during a charge measurement window, which has a charge measurement time period; and deactivate the apparatus for obtaining the charge measurement from the biological tissue and activate the microwave signal generator during a treatment window, which has a treatment time period. Microwave radiation can produce a large amount of noise in a measurement signal being taken from the same location. By alternating the activation of the microwave generator and the measurement system, the accuracy of the measurements obtained may be improved. The measurement window may comprise one or more of: the active mode, which may include the first active mode and/or the second active mode; the sensing ground mode; the reference ground model; and the error compensating mode. The measurement window may comprise multiple sequences of the operation modes described above. The measurement time period may comprise one or more of: the active time period, the sampling period, the time period for the reference ground mode and the time period for the error compensating mode. The measurement time period and the treatment time period may have the same length or may be different. In a specific example, the measurement time period may be 200 ms and the treatment time period may be 400 ms.

The processor is further adapted to automatically deactivate the microwave signal generator based on the determined condition of the biological tissue. In particular, in the case where the biological tissue is a vessel to be sealed, the processor may be adapted to automatically deactivate the microwave signal generator if the vessel is determined as being sealed. In this way, excessive application of microwave radiation to a biological tissue may be avoided. The system may therefore automatically prevent further unnecessary exposure to microwave radiation when the task of sealing the vessel has been completed. Further, the processor may be adapted to generate an alarm signal if the vessel is not determined as being sealed within a predetermined time period. The predetermined time period may be 5 seconds. The system may comprise a user interface adapted to receive the alarm signal and generate an alert to be provided to a user of the system, such as a clinician. The user interface may comprise one or more of: an audio output, wherein the alarm signal is adapted to cause the audio output to generate an audio signal as the alert; a visual output, wherein the alarm signal is adapted to cause the visual output to generate an visual signal as the alert; an haptic output, wherein the alarm signal is adapted to cause the haptic output to generate an haptic signal as the alert; and the like.

Further, the processor may be adapted to generate an alert signal based on the determined condition of the biological tissue. For example, the processor may be adapted to generate an alert signal if the biological tissue is determined to be sealed. The system may comprise a user interface adapted to receive the alert signal and generate an alert to be provided to a user of the system, such as a clinician. The user interface may comprise one or more of: an audio output, wherein the alert signal is adapted to cause the audio output to generate an audio signal as the alert; a visual output, wherein the alert signal is adapted to cause the visual output to generate an visual signal as the alert; an haptic output, wherein the alert signal is adapted to cause the haptic output to generate an haptic signal as the alert; and the like. The user may choose to continue the treatment of the tissue after the alert has been generated.

According to a second aspect, there is provided an electrosurgical apparatus for sensing a condition of a biological tissue. The apparatus comprises a feed structure comprising a microwave channel for connecting a microwave signal generator to a probe for delivering microwave radiation into the biological tissue. The apparatus further comprises a microwave signal detector adapted to: sample reflected power on the microwave channel in response to conveying the microwave radiation to the biological tissue; and generate, from the sampled reflected power, a microwave detection signal indicative of the microwave power reflected by the tissue.

Put another way, there is provided a means of measuring a condition of a biological tissue using the microwave power reflected by the biological tissue, the reflection of the microwave radiation being caused by impedance mismatch between the probe and the biological tissue, as the microwave radiation is being provided to the tissue in order to treat it. Thus, there is provided a means of simultaneously treating a biological tissue using microwaves and monitoring the condition of said tissue during treatment using the same apparatus. The delivery of microwave radiation to a biological tissue will cause the structure of the biological tissue to change, which will in turn change the impedance of the biological tissue and so will change the relationship between the impedance of the probe and the impedance of the tissue. As the reflected microwave power is indicative of this relationship, i.e., the impedance mismatch between probe and tissue, the microwave detection signal obtained from the sampled reflected power is indicative of the condition of the biological tissue.

The feed structure may be any suitable structure for conveying the microwave power to the biological tissue and the microwave power reflected from the biological tissue. The feed structure and microwave channel may comprise one or more of: a cable assembly, such as a coaxial cable; and a waveguide assembly.

The microwave signal detector may be any signal detection circuit capable of obtaining a microwave detection signal from the sampled reflected microwave power. The microwave detection signal may comprise one or more of an amplitude of the sampled reflected power and a phase of the sampled reflected power. Accordingly, the microwave signal detector may be arranged to obtain microwave signal information that is indicative of the magnitude and/or phase of reflected microwave EM radiation.

The microwave signal detector may be further adapted to: sample forward power on the microwave channel in response to conveying the microwave radiation to the biological tissue; and generate, from the sampled forward power and the sampled reflected power, the microwave detection signal indicative of the microwave power reflected by the tissue. Accordingly, the microwave signal detector may be arranged to obtain microwave signal information that is indicative of the magnitude and/or phase of both the microwave EM radiation provided to, and reflected by, the biological tissue. In this case, the microwave detection signal may comprise one or more of a ratio of an amplitude of the sampled forward power to an amplitude of the sampled reflected power and a phase shift between the sampled forward power and the sampled reflected power.

The apparatus may comprise a directional coupler in communication with the microwave channel for sampling the forward power on the microwave channel and the reflected power on the microwave channel. The apparatus may comprise a pair of directional couplers, one for sampling the forward power on the microwave channel and the other for sampling the reflected power on the microwave channel. Any suitable direction coupler may be used.

The apparatus may further comprise a circulator for receiving the microwave radiation to be delivered to the biological tissue and for providing the microwave radiation onwards towards the biological tissue for tissue treatment. The circulator may also be adapted to receive the reflected microwave radiation and direct it towards a termination load, thereby protecting the circuitry of the apparatus form the reflected power. Accordingly, the circulator may be provided at some point along the microwave channel and may be adapted to direct incoming microwave power towards its desired destination. The directional couplers may be provided upstream and/or downstream of the circulator.

The apparatus may further comprise a compensation unit adapted to correct (e.g., error correct) the microwave detection signal. The compensation unit comprises a combiner for combining the sampled reflected power and the sampled forward power. The compensation unit further comprises one or more of: a phase shifter adapted to impart a phase shift on the sampled forward signal prior to the combining; and a variable attenuator, such as a variable resistor, adapted to adjust an amplitude of the sampled forward signal prior to the combining. Accordingly, the sampled reflected power may be corrected using an adjusted version of the sampled forward power. The compensation unit may be a feed-forward compensation network.

According to a further aspect, there is provided an electrosurgical system for treating a biological tissue, the electrosurgical system comprising: an electrosurgical apparatus as described above; and a microwave signal generator adapted to generate the microwave radiation to be delivered to the tissue. The microwave signal generator may comprise any microwave frequency source. The microwave signal generator may comprise an amplification unit adapted to receive a pre-amplified microwave signal from the microwave frequency source and generate the microwave radiation to be provided to the biological tissue by amplifying the pre-amplified microwave signal. The amplification unit may comprise any number of amplifiers.

In an embodiment, the microwave signal generator may be an electrosurgical generator, for example, as disclosed in WO 2012/076844.

The system may further comprise a probe having a distal tip portion for delivering the microwave radiation into the biological tissue and for obtaining the microwave power reflected by the biological tissue, wherein the probe is in communication with the feed structure. The probe may comprise an antenna to radiate the microwave radiation. The antenna may comprise a pair of electrodes provided at the distal tip portion of the probe, such as an inner conductor and an outer conductor of a coaxial cable. In an embodiment, the probe is configured to deliver radiofrequency electromagnetic (EM) energy and/or microwave EM radiation separately or simultaneously from a distal end thereof. In an embodiment, a suitable probe may be as described in WO2011010086A1.

The electrosurgical system may further comprise a processor in communication with the apparatus. The processor may be adapted to: receive the microwave detection signal from the apparatus; and determine a condition of the biological tissue based on the received microwave detection signal. For example, the processor may be adapted to determine if the tissue has been treated based on the microwave detection signal. The processor may be adapted to determine the condition of the biological tissue based on the microwave detection signal using a look-up table, a user defined condition, a neural network, and the like. The processor may be adapted to determine a complex impedance of the biological tissue based on a comparison between a sampled forward power and a sampled reflected power in response to conveying the microwave radiation to the biological tissue.

The biological tissue may be a vessel and the treatment being applied may be sealing the vessel, in which case the processor may be adapted to determine when the vessel has been sealed based on the microwave detection signal. For example, a change in the microwave detection signal may be used to determine that the vessel has been sealed. In a further example, the measurement of the microwave detection signal being greater than or less than a predetermined threshold may be used to determine that the vessel has been sealed. Further, a rate of change of the microwave detection signal crossing (e.g., exceeding) a predetermined value may also be used to determine that the vessel has been sealed. Further, the rate of change of the microwave detection signal approaching a steady state may also be used to determine that the vessel has been sealed.

The biological tissue may also be any other type of tissue undergoing treatment, and in particular undergoing treatment involving the delivery of energy to the biological tissue. The delivery of energy to a biological tissue causes the structure of the biological tissue to change, which in turn changes the impedance mismatch between the probe and the biological tissue. For example, the biological tissue may be a tissue being resected, desiccated, ablated and the like. For example, the biological tissue may be a tumour undergoing ablation.

The processor is further adapted to automatically deactivate the microwave signal generator based on the determined condition of the biological tissue. In particular, in the case where the biological tissue is a vessel to be sealed, the processor may be adapted to automatically deactivate the microwave signal generator if the vessel is determined as being sealed. In this way, excessive application of microwave radiation to a biological tissue may be avoided. The system may therefore automatically prevent further unnecessary exposure to microwave radiation when the task of sealing the vessel has been completed. Further, the processor may be adapted to generate an alarm signal if the vessel is not determined as being sealed within a predetermined time period. The predetermined time period may be 15 seconds. The system may comprise a user interface adapted to receive the alarm signal and generate an alert to be provided to a user of the system, such as a clinician. The user interface may comprise one or more of: an audio output, wherein the alarm signal is adapted to cause the audio output to generate an audio signal as the alert; a visual output, wherein the alarm signal is adapted to cause the visual output to generate an visual signal as the alert; an haptic output, wherein the alarm signal is adapted to cause the haptic output to generate an haptic signal as the alert; and the like.

According to a third aspect, there is provided a system for monitoring a biological tissue, the system comprising: the apparatus for obtaining a charge measurement as described above; and the electrosurgical apparatus for obtaining a microwave detection signal as described above. There is therefore provided a means of sensing the condition of a biological tissue by obtaining a measurement of the charge stored in the tissue and/or by monitoring the microwave power reflected by the tissue.

Such a system may utilise both apparatuses simultaneously, sequentially or individually. The condition of the biological tissue may be sensed based on both of the charge measurement and the microwave detection signal or based on either the charge measurement or the microwave detection signal. The sensing of the condition of the biological tissue using the charge measurement may be used to check the accuracy of the microwave detection signal, and vice versa. Accordingly, such a system is provided with measurement redundancy in case one of the measuring methods ceases to function.

Such a system may use a common shared microwave signal generator (or electrosurgical generator), probe and processor to perform all of the functions described above.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a schematic representation of an apparatus according to an aspect of the invention.

FIG. 2A shows a timing plot of voltage against time representing the switching of the operation modes of the control circuit shown in FIG. 1.

FIG. 2B shows a graph illustrating the frequency response of different tissue types.

FIG. 2C shows a flow chart illustrating a control method using the apparatus of the invention.

FIG. 3 shows an example of an apparatus according to a further aspect of the invention having a differential amplifier arrangement.

FIGS. 4A to 4E show examples of apparatuses according to further aspects of the invention.

FIG. 5 shows an electrosurgical system for treating a biological tissue according to an aspect of the invention.

FIG. 6 shows a flow diagram illustrating a method for using the system shown in FIG. 5.

FIG. 7 shows a plot illustrating the charge measurement for a vessel undergoing a microwave based sealing treatment.

FIG. 8 shows a plot illustrating the charge measurement for a vessel undergoing a microwave based sealing treatment.

FIG. 9 shows a schematic diagram of an electrosurgical apparatus.

FIG. 10 shows an electrosurgical system including an apparatus for sensing a condition of a biological tissue.

FIGS. 11 to 14 show several embodiments of the detection and protection circuit in FIG. 10.

FIG. 15 shows a flow diagram illustrating a method for using the system shown in FIG. 10.

FIG. 16 shows a plot showing the application of microwave radiation to the biological tissue.

FIG. 17 shows a schematic representation of a lumped element model used to model biological tissue and instrument frequency responses.

FIG. 18 shows a graph showing the frequency response of different lumped element models of biological tissues.

FIG. 19 shows a graph showing the frequency response of different lumped element models of system components.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The invention provides an apparatus for obtaining a measurement from a biological tissue for sensing a condition and/or type of the biological tissue, the apparatus comprising: a control circuit, wherein the control circuit comprises an electrical connection for delivering electrical signals to the biological tissue, and wherein the control circuit is adapted to: deliver an electrical signal to the biological tissue for an active time period; and obtain a measurement of an amount of charge stored in the biological tissue in response to the electrical signal during a sampling period after the active time period.

In other words, there is provided an apparatus for obtaining a measurement for monitoring the condition of a biological tissue. The apparatus includes a means of imparting an electrical signal to the biological tissue, in order to provide a charge to the biological tissue, and of measuring the amount of imparted charge stored in said tissue.

Put another way, there may be provided a means of obtaining a measurement representative of the amount of charge that a biological tissue is capable of storing in response to the application of an electrical signal.

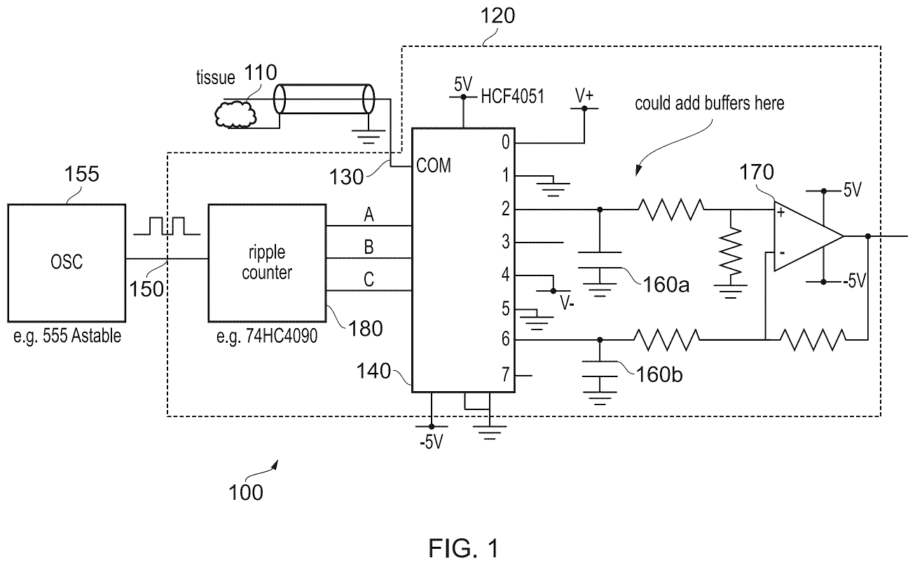

FIG. 1 shows a schematic representation of an apparatus 100 according to an aspect of the invention. In particular, FIG. 1 shows a schematic representation of an apparatus 100 for obtaining a measurement from a biological tissue 110 for sensing a condition and/or type of the biological tissue.

The apparatus 100 comprises a control circuit 120. The control circuit comprises an electrical connection 130 for delivering electrical signals to the biological tissue 110. The control circuit is adapted to deliver an electrical signal to the biological tissue for an active time period and obtain a measurement of an amount of charge stored in the biological tissue in response to the electrical signal during a sampling period after the active time period.

In the example shown in FIG. 1, the apparatus 100 comprises a switching unit 140 adapted to switch the control circuit 120 between a plurality of operation modes. In the specific example shown in FIG. 1, the switching unit is a multiplexer, and in particular a 1:8 multiplexer. The switching unit is adapted to connect the electrical connection 130 to an electrical signal source in an active mode during the active time period. The switching unit may connect the electrical connection to a first electrical signal source during a first active mode and a second, different, electrical signal source during a second active mode. The switching unit is also adapted to connect the electrical connection 130 to a sensing ground in a sampling mode during the sampling period, which follows the active period.

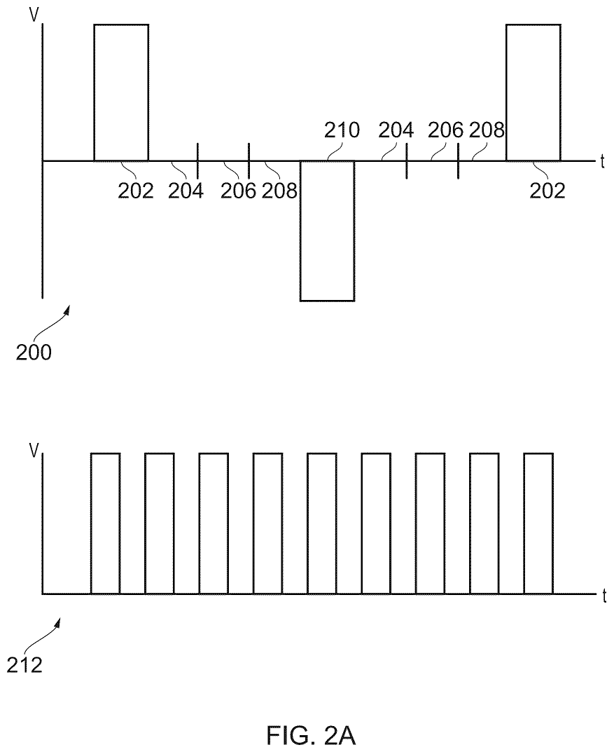

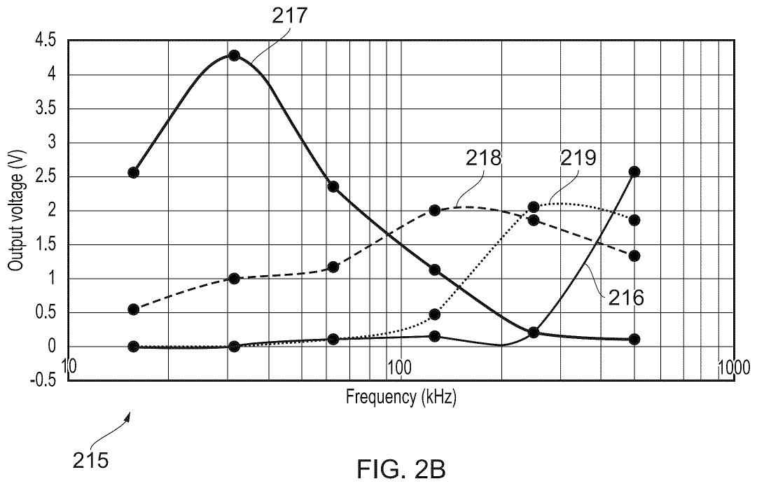

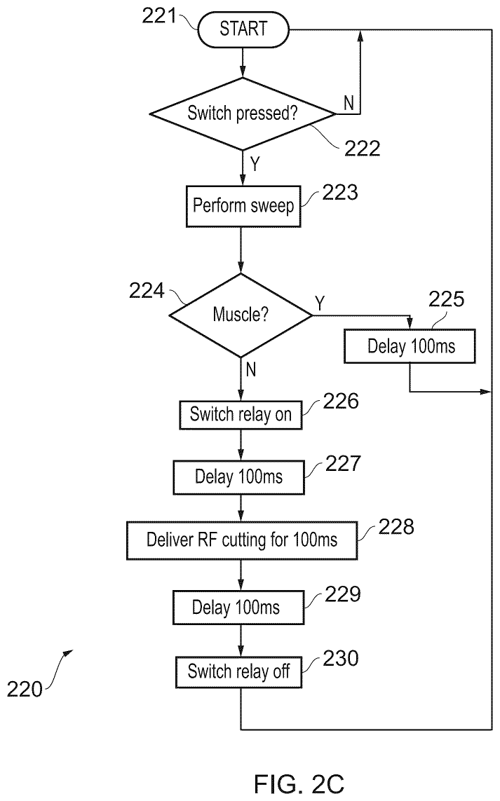

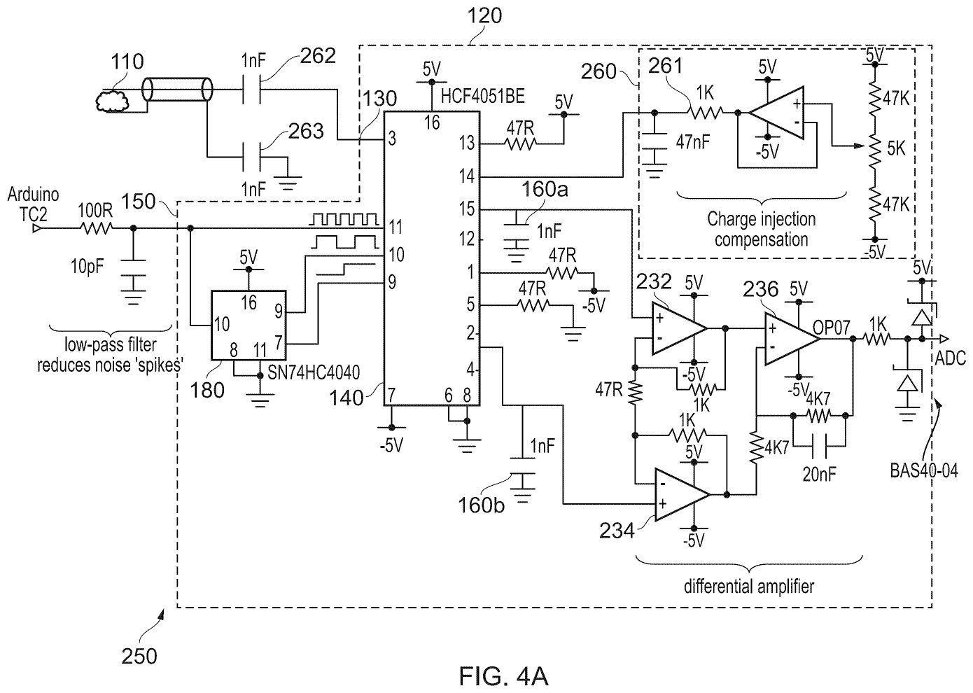

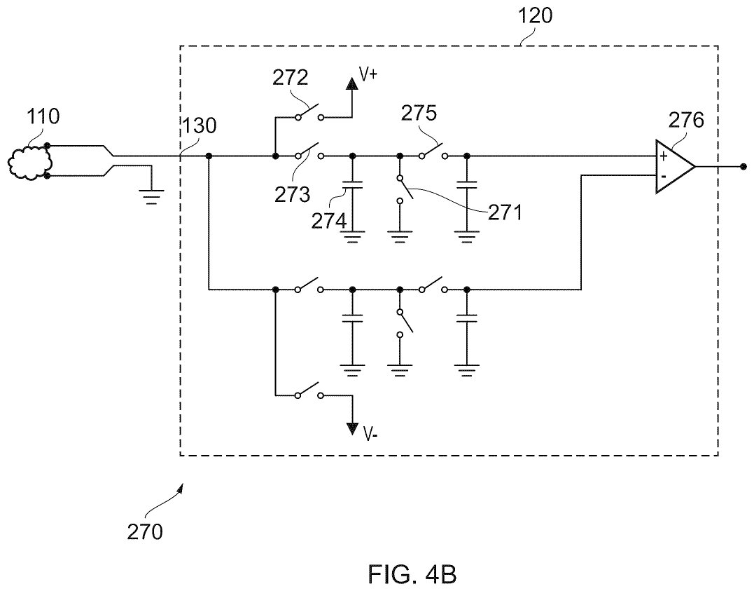

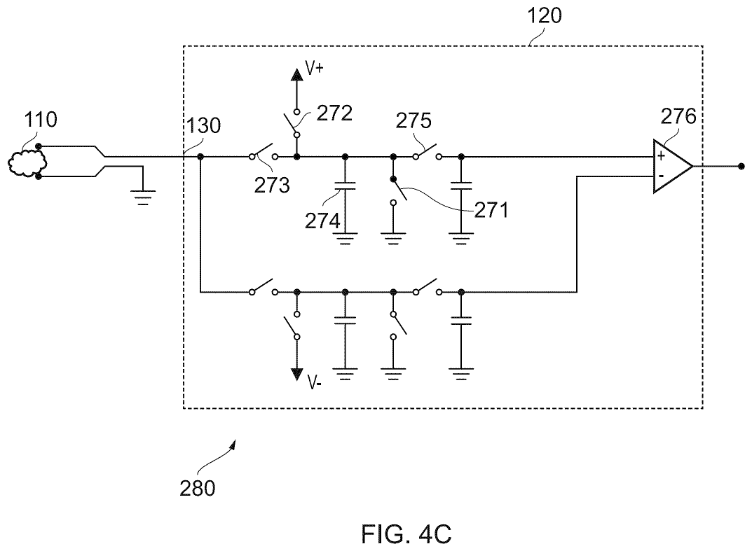

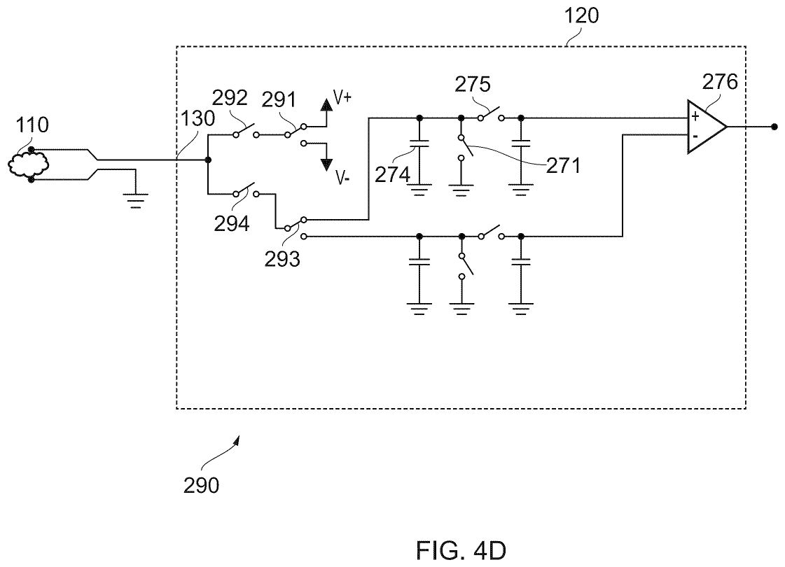

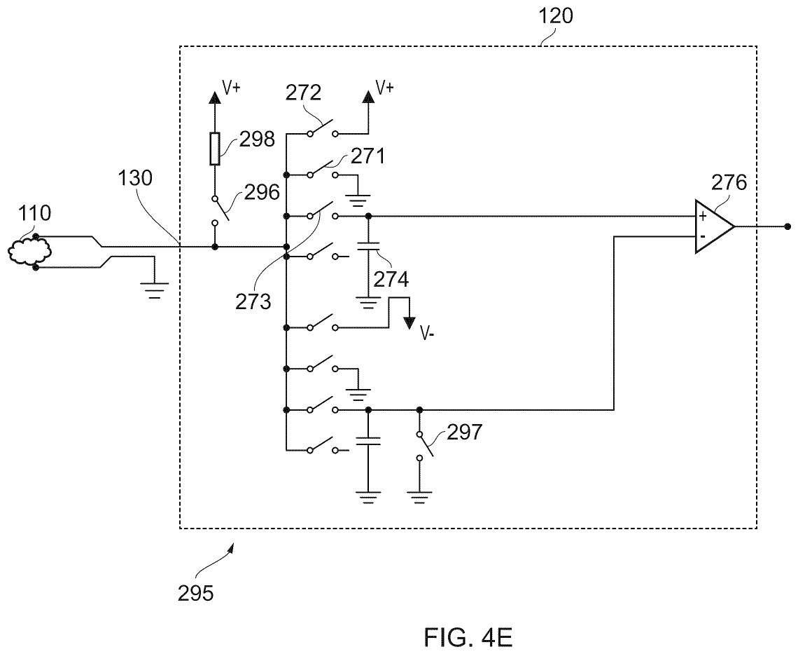

The switching unit 140 may also be adapted to connect the electrical connection to a reference ground during a reference ground mode operation mode. An example of the timing and sequence of the operation modes is discussed in further detail below with respect to FIG. 2. The switching unit 140 may also be adapted to connect the electrical connection to an error compensating circuit during an error compensating mode, which is described in further detail below with reference to FIG. 4.

The apparatus 100 further comprises an input 150 receiving a control signal. The control signal may be generated by an oscillator 155, such as a 555 astable oscillator. The active time period of the active mode may be based on the control signal. In the example shown in FIG. 1, the control signal acts as an input to the switching unit 140, i.e., the multiplexer, to select which of ports 0 to 7 are connected with the common input/output (COM), and so connected with the electrical connection 130 between the control circuit 120 and the tissue 110. For example, the control signal may be a square wave signal with a frequency between 100 Hz and 100 MHz. In this case, the multiplexer may be adapted to cycle through ports 0 to 7 in response to the rising or falling edge of the square wave control signal.

In practise, in a first instance, port 0 may be connected to the COM port, meaning that the electrical connection 130 is connect to a positive voltage source, which may be a +5V DC voltage source. In this state, the apparatus is imparting a charge on the biological tissue. The biological tissue may take on some of the charge and store it, in a similar manner to a capacitor. The amount of charge stored in the biological tissue can vary according to tissue type; however, for a specific tissue, the amount of charge able to be stored in said tissue will also vary based on the condition of said tissue. In the case where the tissue is a vessel being sealed by the application of microwave energy, the amount of charge able to be stored in the vessel tissue at the sealing point will change as the delivery of the microwave energy changes the structure of the tissue during the sealing treatment. This applies equally to any tissue undergoing treatment by the delivery of energy.

The control circuit 120 maintains this connection until the control signal triggers the multiplexer to change the connection to the next port. In the examples described herein, the switching unit 140 changes the operation mode of the control circuit, for example by switching which port of the multiplexer is connected to the COM port, in response to the rising edge of a square wave control signal. Put another way, the multiplexer is triggered on the rising edge of the control signal. However, it will be appreciated that the falling edge of the control signal or both the falling and rising edges may equally be used for the same purpose.

Accordingly, port 0 remains connected to the COM port until the next rising edge of the control signal arrives at the switching unit. In this way, the active time period, i.e., the length of time the control circuit is in the active mode maintaining a connection between the electrical connection 130 and the electrical signal source, is dictated by the control signal. More specifically, the active time period is based on the period of the control signal and in the particular example shown in FIG. 1, the active time period is half the period of the control signal, meaning a control signal of 500 kHz will result in a 1 μS active time period.

When the next rising edge arrives at the switching unit 140, the multiplexer switches the connection to be between port 1 and the COM port. In FIG. 1, port 1 is connected to a reference ground, meaning that the control circuit will enter the reference ground mode. In this mode the electrical connection 130, and so the biological tissue 110, is connected to the reference ground. This will cause any surface charges on the biological tissue to be removed, but will not remove the charge stored within the biological tissue. Accordingly, the subsequent measurement of the amount of charge stored in the tissue will be more representative of the true value of the amount of charge stored in the tissue as the surface charges will not be present in the measurement. Thus, the accuracy of the charge measurement will be improved.

Once again, port 1 will remain connected to the COM port until the next rising edge of the control signal arrives as the switching unit. In this way, the reference ground period will be the same length of time as the active time period, which may be the half period of the control signal as described above.

At the next rising edge of the control signal, the multiplexer switches the connection to be between port 2 and the COM port. In FIG. 1, port 2 is connected to a sensing ground, meaning that the control circuit will enter the sensing ground mode. In this mode the electrical connection 130, and so the biological tissue 110, is connected to a sensing ground, which will cause the charge stored in the tissue to be released so that the stored charge can be collected and sampled in order to obtain the charge measurement. In the example shown in FIG. 1, the sensing ground is connected to a sampling capacitor 160a, which may for example be a 1 nF capacitor, for collecting the charge measurement from the biological tissue. The sensing ground is also connected to an amplifier circuit 170, which is described in further detail below.

Once again, port 2 will remain connected to the COM port until the next rising edge of the control signal arrives at the switching unit. In this way, the sampling period will be the same length of time as the active time period and the reference ground period, which may be the half period of the control signal as described above.

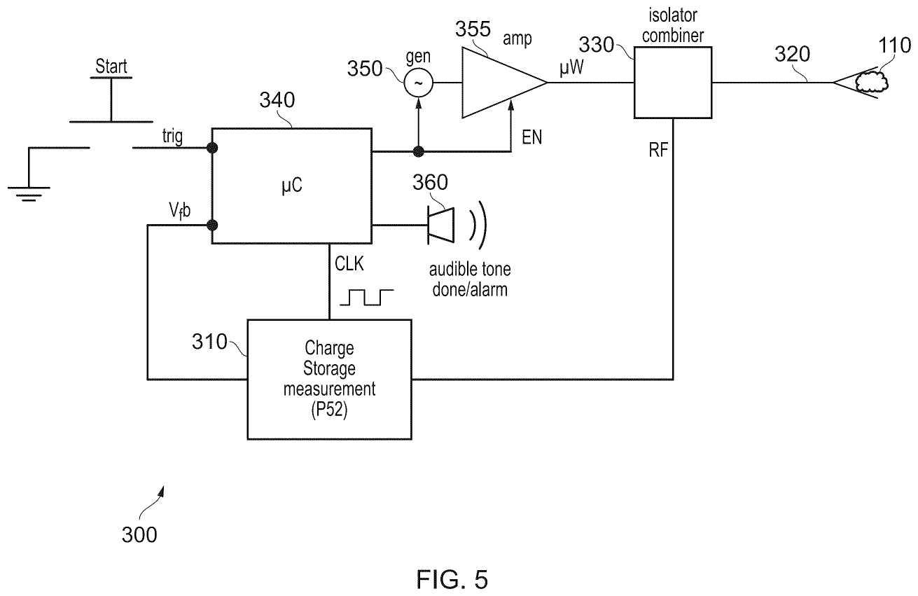

At the next rising edge of the control signal, the multiplexer switches the connection to be between port 3 and the COM port. In FIG. 1, port 3 is disconnected at a high impedance. Accordingly, the control circuit enters what may be referred to as a rest mode where no electrical signals pass to or from the biological tissue 110. However, during this time period, the sampling capacitor 160a, which was charged by the biological tissue 110 in the sensing ground mode, may discharge through the amplifier circuit 170 and be passed to a processor for determining the condition and/or type of the biological tissue based on the measurement, which is described in further detail below with reference to FIG. 5.

At each subsequent rising edge of the control signal, the multiplexer will switch the connection to be between the COM port and ports 4, 5, 6 and 7 in turn. The behaviour of the control circuit when connected to ports 4, 5, 6 and 7 will mirror its behaviour when connected to ports 0, 1, 2 and 3, respectively. The exception to this is port 4, which connects the COM port to an electrical signal source having the opposite polarity to the source connected to port 1. Therefore, when the COM port is connected to port 0, the control circuit is in the first active mode and when the COM port is connected to port 4, the control circuit is in the second active mode. For example, port 4 may connect the COM port to a −5V DC voltage source. In all other respects, the behaviour of the control circuit when the COM port is connected to ports 4, 5, 6 and 7 is the same as when connected to ports 0, 1, 2 and 3, respectively. Once the multiplexer reaches port 7, the next rising edge will cause the multiplexer to reconnect the COM port with port 0 and the sequence will begin again as described above.

In the example shown in FIG. 1, the control circuit 120 further comprises a counter 180, which may for example be a ripple counter, adapted to generate a clocking signal based on the control signal. The ripple counter may comprise any number of flip-flop circuits connected in series. Accordingly, the switching unit 140 may be adapted to cycle the control circuit through the plurality of operation modes described above based on the clocking signal. The clocking signal may comprise the control signal itself, which may be provided to the multiplexer on line A. The clocking signal may further comprise a first clock signal having a first clock signal period, the first clock signal period being different to the control signal period. The first clock signal may be provided to the multiplexer on line B. The clocking signal may further comprise a second clock signal having a second clock signal period, the second clock signal period being different to the control signal period and the first clock signal period. The second clock signal may be provided to the multiplexer on line C. Accordingly, the multiplexer may receive up to three different clocking signals at registration ports in order to select which port to connect to the COM port.

The control signal may be swept between a plurality of frequencies. For example, the frequency of the control signal may be altered after each sequence of operational modes has completed. By way of a specific example, the control circuit may be cycled through a first active mode, an error compensating mode, a floating mode, a second active mode, a reference ground mode and a floating mode using a control signal at 500 kHz in a first sequence. The control circuit may then be cycled through the same sequence of operational modes using a control signal at 32 kHz in a second sequence. The control signal may then be switched alternately between the two frequencies. Thus, charge measurements at two, or more, different frequencies may be obtained from the biological tissue in the same measurement operation. The charge measurement obtained at the first frequency and the charge measurement obtained at the second frequency may be used in combination, or separately, to sense the condition and/or type of the tissue. The charge measurements obtained at different frequencies may exhibit different behaviours as the structure of the biological tissue changes in response to treatment. For example, when the charge measurement is obtained at 500 kHz, the charge measurement may approach 0V when the vessel is sealed; whereas, when the charge measurement is obtained at 32 kHz, the charge measurement may rapidly increase when the vessel is sealed. Accordingly, different thresholding conditions may be applied to the charge measurements obtained at different frequencies for sensing the condition of the tissue. The measurement may also be performed at more than two frequencies and each measurement may be used in combination with the other measurement or separately for sensing the condition of the tissue. The relative change of the charge measurement between different frequencies may be used as an indicator of tissue type due to dielectric relaxation effects in different tissues.

Using the charge measurements at two different frequencies, the sealing of a vessel, or other tissue, may be detected when the detected charge stored at the two frequencies crosses over each other, as tissue can store charge over a longer time when sealed. The sensitivity of this method of detecting tissue sealing may be adjusted in a number of ways.

One way in which the sensitivity of detecting tissue sealing, based on the intersection of measured tissue charge storage at different frequencies, may be increased is to vary the measurement frequencies used to determine the crossover sealing point. In particular, adjusting the lower of the two frequencies allows for a greater resolution of frequency adjustment, and thereby a greater resolution of sensitivity adjustment. However, in some cases, such as apparatuses using external oscillators, for example voltage-controlled oscillators, either frequency could be adjusted.

Put another way, the charge storage measurement of the biological tissue may be taken at a number of different frequencies, such as two different frequencies, to monitor how the peak frequency of the tissue being sealed changes as a means of monitoring the progression of the sealing process.

Typically, the charge storage for unsealed tissue has a much higher peak frequency than that of sealed tissue. For example, a sealed tissue may have a peak frequency of approximately 32 kHz or below, and the unsealed tissue may have a peak frequency above 250 kHz. Thus, at the optimal moment of seal, the frequency at which the greatest charge storage occurs falls, thereby indicating that sealing has occurred. By changing the compared frequencies whilst measuring charge stored by the tissue, the peak frequency at which sealing is stopped may be varied. A lower frequency which is closer to the higher frequency will result in the seal being stopped sooner because the peak frequency has decreased less, whereas a lower frequency which is further away from (e.g. significantly lower than) the higher frequency will result in the seal being stopped later because the peak frequency has to fall further for the crossover trigger point to be reached.

Each different tissue type may have different peak frequencies and be associated with different cutting tolerances (e.g., associated with risks of over-cutting or under-cutting the biological material). Accordingly, the upper frequency may be selected based on the tissue type known to be contacting the instrument, which may be identified as described in detail below, and a known peak frequency of the sealed state of said tissue and the lower frequency may be selected according to a tolerance level associated with cutting or sealing the given tissue and its surrounding tissue types.

Alternatively, or in addition to the frequency adjustment described above, the sensitivity of detecting tissue sealing may be adjusted by applying a scaling factor to the measured signal at one or both measurement frequencies.

One of the measured frequencies may be scaled relative to the other before they are compared, for example, stopping sealing when the measurement at 31.25 kHz is greater than half of the measurement at 500 kHz, rather than simply greater than it, would result in a slightly earlier shut-off. In an application in which identification of tissue types with a similar frequency response is desirable (for example liver and muscle), such fine-tuning using a scaling factor may be useful. Sealed/unsealed measurements of one known tissue type (e.g. vessel) may be used to form a look-up table, or different (unsealed) tissue types may be differentiated from each other as the frequency response changes observed are similar.

In the example shown in FIG. 1, the control circuit 120 comprises an amplifier unit 170. The amplifier unit 170 shown in FIG. 1 is an operational amplifier and receives the charge measurement obtained following the first active mode, when the COM port was connected to port 0, and the charge measurement obtained following the second active mode, when the COM port was connected to port 4. The amplifier unit takes the difference between the two measurements in order to obtain a final charge measurement for use in sensing the condition and/or type of the tissue 110. In this way, the signal to noise ratio of the final charge measurement is doubled, as the amplitude of the measured differential voltage is doubled in comparison to measuring only positive or negative, thereby improving the accuracy of the final charge measurement. Alternatively, either measurement from the first or second active modes may be utilized in isolation.

FIG. 2 shows an example timing plot 200 of voltage against time, which represents the switching of the operation modes of the control circuit for the apparatus shown in FIG. 1. FIG. 2 also shows a plot of a control signal 212 corresponding to the timing plot 200, wherein the operation modes are switched on the rising edge of the control signal.

The order of the operation modes illustrated in the timing plot is a first active mode 202, a reference ground mode 204, a floating mode 206, a resting mode 208, a second active mode 210, a reference ground mode, a floating mode, a resting ground mode. This is one complete sequence and then the operation mode returns to the first active mode as the next sequence begins.

As outlined above, tissue type, and boundaries between tissue types, may be derived from the measurements in order to adjust the control of the electrical signals based on the tissue type in contact with the electrical connection.

FIG. 2B shows a logarithmic graph 215 of output voltage, as measured from a tissue sample, against the frequency of the operation mode switching. The graph shows four different plots illustrating the different frequency responses of different tissue samples and contains some example typical frequencies of interest.

Plot 216 represents the frequency response of a muscle tissue sample. As shown in FIG. 2B, the frequency response of muscle is minimal until the frequency approaches 500 kHz, where the response approaches a peak. The peak frequency for muscle tissue is possibly greater than 500 kHz, this being the highest frequency available in the testing apparatus for obtaining the data shown in FIG. 2B; however, the data is sufficient for illustrating the following points regarding differentiating between different tissue types.

Plot 217 represents the frequency response of a fat tissue sample. As shown in FIG. 2B, the frequency response of fat peaks at roughly 31 kHz, above which the response decays to a minimal level as the frequency approaches 500 kHz.

Comparing plots 216 and 217, it will be appreciated that the frequency response of the charge storage of the tissue being measured provides a clear mechanism for differentiating between the muscle and fat. For example, if the measured signal is greatest at 500 kHz, muscle is indicated. If the response is greatest at 31.25 kHz, fat is indicated.

Plot 218 represents the frequency response of a submucosa tissue sample. As shown in FIG. 2B, the frequency response of submucosa peaks between 100-200 kHz. Plot 219 represents the frequency response of a liver tissue sample. As shown in FIG. 2B, the frequency response of liver peaks between 250-350 kHz.

The measurements illustrated in FIG. 2B were obtained using two needle electrodes of tinned copper wire approximately 0.5 mm diameter and 5 mm in length, pushed against the surface of the tissue with force almost sufficient to penetrate. The conditional statements which may be used to make a decision on tissue type in contact with the electrode may comprise identifying the frequency at which the largest signal was obtained, with different peak frequencies identifying different tissue types. Sealed and unsealed measurements of one known tissue type (e.g. vessel) may be used to form a look-up table, then charge storage measurements may be compared to values in that look up table to identify which tissue is being treated and/or when sealing happens. Optionally, a tissue type may be known or provided (e.g. by a user) and the look up table could be used only as part of a mechanism for determining when sealing happens.

If it is desirable to indicate a shut-off for muscle, an upper peak frequency can be set above which cutting is prevented or an alarm is sounded. For example, an upper peak frequency of 250 kHz was found to work well in testing. In this case the lower the frequency, the more often cutting was prevented.

It should be noted that the closer the peak frequencies of the different tissue types, the harder it is to differentiate between them. For example, cutting fat and not muscle is very easy due to the large difference in frequency responses of the two tissue types; whereas, cutting liver but not muscle is more challenging. The decision point can be fine-tuned by applying scaling factors to the measurements at each frequency, however a peak frequency decision point of 250 kHz was found to work acceptably for preventing cutting through the oesophagus muscle wall such that cutting was allowed only if the peak frequency was below 250 kHz.

The frequency response of the tissue in contact with the probe is tested over a range of frequencies by performing a frequency sweep of a range of frequencies of interest (between 10 kHz and 500 kHz in the example shown in FIG. 2B; however, any suitable range of frequencies may be utilized for such a frequency sweep).

In the example shown in FIG. 2B, the frequency sweep is performed every 1 second, but a sweep can be performed far more frequently if desired. For the demonstration of not cutting oesophagus muscle wall, the sensing was done at approximately 100 ms intervals, or 300 ms intervals if cutting was allowed (due to the additional time taken to switch over to RF power delivery using a relay, and the time for which the RF power delivery is active).

It should be noted that in the case where no charge storage response is measured for any frequency, it may be determined that the probe is not in contact with any tissue or that there is a fault present within the apparatus. Accordingly, this result may be used to prevent damaging energy delivery from occurring when the probe is not in tissue or a fault is present within the system.

FIG. 2C shows a flow diagram 220 illustrating a control method for controlling the delivery of RF cutting energy based on tissue detection. In particular, the method shown in FIG. 2C illustrates a control scheme for preventing the cutting of muscle tissue by the probe.

The method begins in step 221 with the initiation of the system, which may for example be the system shown in FIG. 1. In step 222, it is determined whether or not the switch for delivering energy to the biological tissue has been pressed. If the switch has not been pressed, the system waits in a standby mode.

When the switch is pressed, the method progresses to step 223 where the charge measurement is performed across a desired range of frequencies in a sweep. The result of such a frequency sweep will be a frequency response plot similar to those shown in FIG. 2B, with a shape characteristic to the tissue being investigated.

Once the frequency sweep has been performed, the method progresses to step 224, where it is determined if the tissue in contact with the probe is muscle tissue, for example, by identifying the peak frequency response from step 223. If it is determined that the probe is in contact with muscle tissue, the method progresses to step 225 to delay any further energy delivery by 100 ms. In addition, an alert may be generated to inform the clinician that the probe is in contact with muscle tissue.

If it is determined in step 224 that the probe is not in contact with muscle tissue, the method may progress to step 226 to activate the relay and, following a 100 ms delay in step 227, deliver RF cutting energy to the biological tissue for 100 ms in step 228.

Following a further 100 ms delay in step 229, the relay may be deactivated in step 230 and the method may return to step 222 in order to perform a looped tissue detection whilst the RF cutting energy is being demanded.

The timings shown in FIG. 2C are not critical and may be adjusted according to the implementation of the system. More frequent sensing reduces the time available for power delivery, and the 100 ms delays shown may provide a balance between tissue sensing frequency, and therefore accuracy, and power delivery time.

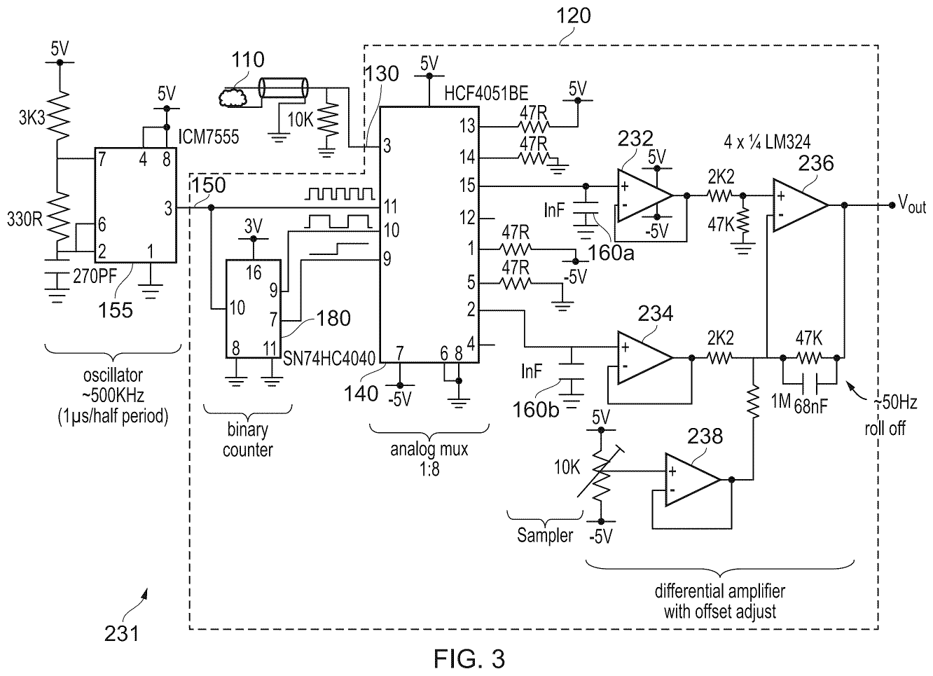

FIG. 3 shows an example of an apparatus 231 according to a further aspect of the invention. Many of the components of the apparatus 231 shown in FIG. 3 are functionally identical to those described above with reference to FIG. 1 and reference numerals have been reused to indicate those components that function in the same way.

In the example shown in FIG. 3, the apparatus comprises a differential amplifier arrangement comprising a first amplifier circuit 232 adapted to amplify the measurement obtained in the floating mode following the first active mode and a second amplifier circuit 234 adapted to amplify the measurement obtained in the floating mode following the second active mode. Further, the differential amplifier arrangement further comprises a third amplifier circuit 236 adapted to obtain the difference between the amplified measurement from the first amplifier circuit and the amplified measurement from the second amplifier circuit.

This arrangement has a high input impedance and so doesn't load the 1 nF capacitors 160a and 160b. The first amplifier circuit 232 and second amplifier circuit 234 act as buffers which provide a low impedance drive signal to the differential amplifier, i.e., the third amplifier circuit 236.

The apparatus 231 may further include an offset adjustment circuit 238 to trim out an observed offset on the charge measurement when no was tissue present, at a single operating frequency of 500 kHz during testing. This offset was traced to a frequency-dependent component due to the charge injection of the analogue switches, and a non-frequency dependent component which was due to the use of certain opamps for the amplification circuits. Accordingly, the offset adjustment circuit 238 may be disregarded in implementations of the invention where this offset does not occur, for example as shown in FIG. 4A.

FIG. 4A shows an example of an apparatus 250 according to a further aspect of the invention. Many of the components of the apparatus 250 shown in FIG. 4A are functionally identical to those described above with reference to FIG. 1 or FIG. 3 and reference numerals have been reused to indicate those components that function in the same way.

In the arrangement shown in FIG. 4A, the apparatus further comprises a charge injection compensation unit 260. The charge injection compensation unit connects to one of the ports of the multiplexer, which in the example shown in FIG. 4 is port 14 which corresponds to port 1 in FIG. 1. By connecting the COM port (port 3 in FIG. 4A) to the charge injection compensation unit, the control circuit enters the error compensating mode, which is provided here as an alternative to the reference ground mode between the first active mode and the sensing ground mode. The charge injection compensation unit provides an offset electrical signal to the tissue in order to provide a small amount of additional charge to the biological tissue 110 in order to correct an error in the charge measurement.

In testing, the source of this error was found to be charge injection in the analogue switches within the analogue multiplexer IC. This is an offset voltage caused by an injection of charge from the voltage change across parasitic capacitances of the MOS devices used to implement each analogue switch during switching, i.e., a fixed charge delivery per cycle, hence why it is a frequency-dependant error. Analogue switches are typically designed to minimise charge injection by balancing the capacitances of the switching devices, but this can only be done at one voltage, usually OV. The trimmable low voltage of the charge injection compensation unit allows the charge injection on the positive measurement to be adjusted, such that the net charge injection from the positive and negative channel cancels and this source of offset is trimmed out. Accordingly, the charge injection compensation unit 260 may be disregarded in implementations of the invention where this charge injection offset does not occur.

Alternatively, or in addition to the methods described above, the sensitivity of detecting tissue sealing may be adjusted by adjustment of the offset trimming voltage. The offset voltage does not influence the state of the tissue at shut-off, but rather allows the threshold for tissue detection to be varied. Adding an offset causes the measurement to be non-zero over the range of frequencies. This allows a variable threshold above which tissue is detected. For example, if a sufficient positive offset is applied, the microcontroller will always detect that tissue is present. If a negative offset is applied, a corresponding amplitude of signal must be present before tissue is detected. This is particularly useful for tissue type identification, to indicate muscle/fat (for example) if tissue is detected as present.

In an alternative arrangement, the trimmer resistor 261 which sets the offset voltage may be replaced by a digital potentiometer. In this way, the microcontroller is configured to perform an auto-zero sequence at start-up, trimming the measurement to zero before any instrument is connected to the generator by adjusting the resistance of the digital potentiometer. Further, the generator may be adapted to self-calibrate, for example by connecting the sensing circuit to one or more pre-set tissue circuit models comprising discrete resistors and capacitors, at start-up or periodically during standby. This may be performed using relays or analogue switches at the input to the sensing circuit.

In the example shown in FIG. 4A, DC blocking capacitors 262 and 263 are provided between the tissue and the system to prevent DC current flow into the patient. In alternative arrangements, rather than using DC blocking capacitors, preventing DC current flow into a patient may be achieved by electrically floating the whole measurement circuit on the secondary side of the patient isolation barrier. This may be performed, for example, by using a suitably rated digital isolator and a local analogue to digital converter or microcontroller on the secondary side of the patient isolation barrier to convert the measured analogue signal into a digital response, which is fed back across the patient isolation barrier to the main generator controller in digitized form.

As will be appreciated, the circuits illustrated in FIGS. 1, 3 and 4A represent only a few implementations of the invention. The concept of the invention may be achieved using a variety of different circuitry and is not limited to the specific circuit components discussed here. Further examples of different circuitry that may be used to achieve the concepts of the invention are shown in FIGS. 4B to 4D.

FIG. 4B shows an example of an apparatus 270 according to a further aspect of the invention. Many of the components of the apparatus 270 shown in FIG. 4B are functionally identical to those described above with reference to FIG. 1, FIG. 3 and/or FIG. 4A and reference numerals have been reused to indicate those components that function in the same way. In the specific example of FIG. 4B, the switching unit is shown in more detail and the control circuitry is not shown in detail for the sake of clarity.

To obtain a charge measurement from the tissue sample 110, the apparatus 270 of FIG. 4B operates as follows, starting from a state where every switch is open. Switch 271 closes. At the beginning of the active mode, switch 272 closes in order to apply voltage V+ to the tissue 110. Switch 272 opens once again after the active mode time has elapsed.

Switch 273 closes at the beginning of the reference ground mode, thereby earthing the surface of the tissue 110 via switch 271. At the end of the reference ground mode, switch 271 opens and the charge stored in the tissue is sampled by capacitor 274 during the measurement period.

Switch 273 then opens and switch 275 closes to provide the sampled voltage to operational amplifier 276, from which the measurement may be passed onto a system for further interpretation. Switch 275 may then be opened.

It will be appreciated that the remaining switching circuitry is the mirror of the circuitry already described for FIG. 4B and will function in the same manner for the application and measurement of voltage V-.

FIG. 4C shows an example of an apparatus 280 according to a further aspect of the invention. Many of the components of the apparatus 280 shown in FIG. 4C are functionally identical to those described above with reference to FIG. 4B and reference numerals have been reused to indicate those components that function in the same way.

Indeed, FIG. 4C differs from FIG. 4B only in that only two of the switches are directly connected to the tissue as opposed to the four switches shown in contact with the tissue in FIG. 4B. In this way, the effect of parasitic capacitance on the charge measurement obtained from the tissue may be reduced by reducing the number of electronic components in direct contact with the tissue.

FIG. 4D shows an example of an apparatus 290 according to a further aspect of the invention. Many of the components of the apparatus 290 shown in FIG. 4D are functionally identical to those described above with reference to FIGS. 4B and 4C and reference numerals have been reused to indicate those components that function in the same way.

In the example shown in FIG. 4D, the switching unit comprises a single pole, double throw (SPDT) switch 291 for switching between the polarities applied to the tissue by way of switch 292. In addition, the switching unit comprises a further SPDT switch 293 for selecting which arm of the sampling portion of the control unit 120 the charge measurement is taken with by way of switch 294.

FIG. 4E shows an example of an apparatus 295 according to a further aspect of the invention. Many of the components of the apparatus 295 shown in FIG. 4E are functionally identical to those described above with reference to FIGS. 4B, 4C and 4D and reference numerals have been reused to indicate those components that function in the same way.

Indeed, the charge measurement using the apparatus 295 of FIG. 4E is substantially identical to the method described in relation to FIG. 4B in terms of switch activation.

The apparatus 295 of FIG. 4E differs from the apparatus 270 shown in FIG. 4B in the provision of additional switch 296 connected to a positive voltage source V+ and switch 297 connected to ground. These two additional analogue switches, and the one additional resistor 298, allow DC resistance to be measured. Closing switches 296, 273 and 297 causes a potential divider to be formed between resistor 298 and the tissue 110, thus causing a DC voltage to appear at switch 296. Resistor 298 could be replaced with a current source to achieve a similar effect. Closing switches 273 and 297 causes this voltage to be fed to the input of the instrumentation amplifier 276.

This DC resistance measurement may be utilized, for example, in detecting liquid contamination of the instrument or failure of the instrument, if the charge storage measurement reads low over a range of frequencies but the measured resistance is low. Detecting a resistance below a preset threshold and a valid stored charge measurement may allow detection of the instrument contacting tissue, as opposed to being covered in blood or residue, or when held in air. This may be used, for example, to cause the generator to deliver a warning or prevent energy delivery when energy is demanded under this condition, reducing the likelihood of instrument damage. The measurement of resistance may also be used in combination with the charge measurements for detection of an effective seal, or for regulating the level of energy being delivered.

With switches 296 and 297 open, the circuit functions as normal to measure stored charge in the tissue. This means, for example, that resistance and stored charge may be measured alternately. This requires DC current to be applied to the tissue, although ‘floating’ the whole measurement circuit on the patient side of the isolation barrier would cause this DC current to only flow locally to the instrument.

In the examples of the circuitry described above, the input from the medical instrument is connected directly to the analogue switches. It is noted that such an arrangement may leave the circuit vulnerable to ESD-induced damage. In a practical implementation there would be ESD protection measures such as transient voltage suppressing or clamping diodes and/or current-limiting resistors on the input to the circuit, which have not been shown in the Figures. These should not affect the behaviour of the circuit, as these devices would not normally conduct. Parasitic capacitance of these example clamping diodes behaves in parallel with the capacitance of the cable, and as the measurement technique is not sensitive to this, the charge measurement is unaffected by the addition of clamping diodes.

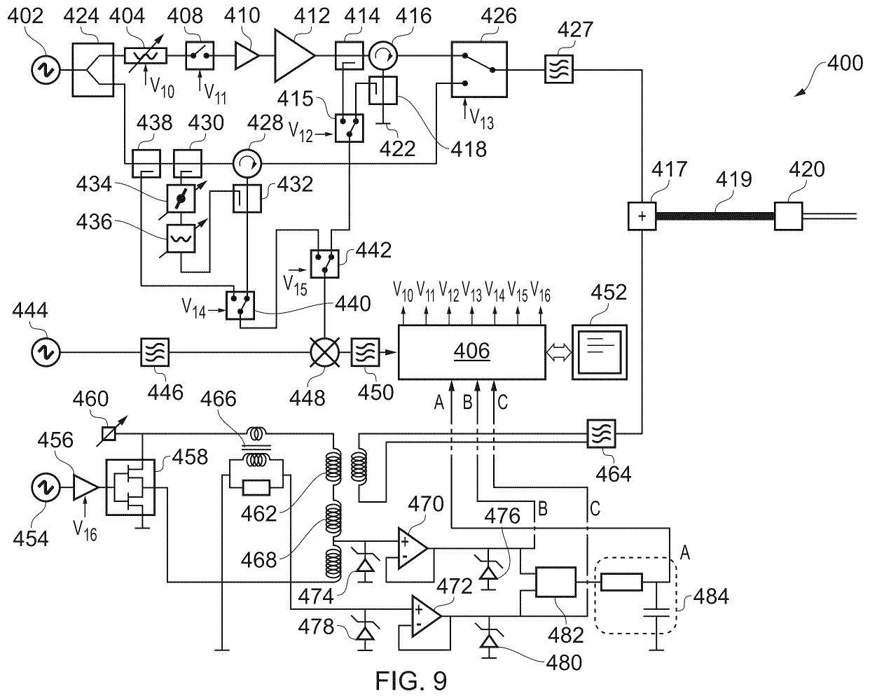

FIG. 5 shows an electrosurgical system 300 for treating a biological tissue 110 according to an aspect of the invention. The electrosurgical system 300 comprises an apparatus 310 as described above, for example in reference to FIG. 1, 3 or 4. The system further comprises a probe 320 having a distal tip portion for delivering the electrical signal into the biological tissue and for obtaining the charge measurement from the tissue. The probe is in electrical communication with the apparatus 320 by way of an isolator combiner circuit 330, which is described in further detail below with respect to FIG. 9. The distal tip portion of the probe comprises a pair of electrodes for contacting the biological tissue 110, which act as an active electrode and a return electrode for the electrical signal being provided to the biological tissue and for obtaining the measurement from the biological tissue.

The electrosurgical system 300 further comprises a processor 340, which may be a microcontroller for example, in communication with the apparatus 310. The processor is adapted to receive the charge measurement from the apparatus and determine a condition and/or type of the biological tissue 110 based on the received charge measurement.

The electrosurgical system 300 further comprises a microwave signal generator 350 for generating microwave EM radiation, for example in combination with an amplification unit 355. The microwave EM radiation is provided to the biological tissue 110 in order to treat the tissue. For example, the microwave EM radiation may be provided to a vessel in order to seal the vessel. Accordingly, in addition to providing the electrical signal to the biological tissue and obtaining the charge measurement from the biological tissue, the probe 320 is further adapted to deliver the microwave electromagnetic radiation to the tissue. The processor is further adapted to activate the apparatus for obtaining the charge measurement from the biological tissue and deactivate the microwave signal generator during a charge measurement window, which has a charge measurement time period. Further the processor is adapted to deactivate the apparatus for obtaining the charge measurement from the biological tissue and activate the microwave signal generator during a treatment window, which has a treatment time period. Deactivating the apparatus may comprise the processor ignoring the signal from the apparatus during the treatment window and the apparatus may run continuously. Thus, the measurement hardware runs continuously, but the processor ignores the signal from it whilst the microwave power is applied and only takes an ADC reading when the microwave is off. Put another way, the processor alternates the activation of the microwave generator and the measurement system.

The processor is further adapted to automatically deactivate the microwave signal generator based on the determined condition of the biological tissue. In particular, in the case where the biological tissue is a vessel to be sealed, the processor may be adapted to automatically deactivate the microwave signal generator if the vessel is determined as being sealed. The system may therefore automatically prevent further unnecessary exposure to microwave radiation when the task of sealing the vessel has been completed. The vessel may be determined to be sealed base on one or more of: the charge measurement crossing a predetermined threshold; a rate of change of the charge measurement; a rate of change of the charge measurement approaching a steady state; and a rate of change of the charge measurement crossing a predetermined threshold. For example, the vessel may be determined to have been sealed when the measurement of the charge stored in the biological tissue is near or at zero. Alternatively, a sudden sharp increase in the amount of charge that can be stored in the biological tissue may also be used to determined that vessel sealing is complete.

Further, the processor may be adapted to generate an alarm signal if the vessel is not determined as being sealed within a predetermined time period. The predetermined time period may be 5 seconds. The system shown in FIG. 5 comprises an audio output 360 for generating an audio signal as the alert.

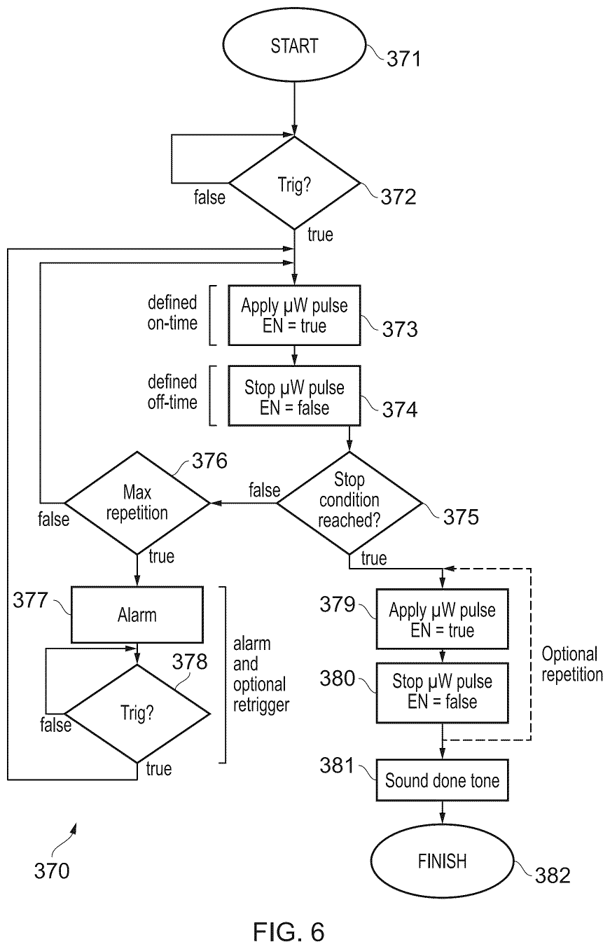

FIG. 6 shows a flow diagram 370 illustrating a method for using the system shown in FIG. 5.

The method begins in step 371 and proceeds to step 372, where it is determined whether the system has been activated for delivering microwave energy to a biological tissue. The system waits until the trigger signal is received and then progresses to step 373, where a microwave pulse is applied to the biological tissue during a treatment window as described above.

After the treatment window has elapsed, the method progresses to step 374 where the microwave pulse is stopped and in step 375 it is determined whether a stop condition has been reached based on the charge measurement, such as the charge measurement falling below a predetermined threshold during a measurement window. If the stop condition has not been reached, the method progresses to step 376 where it is determined whether a maximum number of repetitions of the method has been reached. In other words, at step 376 it is determined whether the vessel has been sealed within a predetermined time or not. If the vessel has not been sealed within the predetermined time, an alarm is sounded 377 and the method may cease or continue from step 378 according to the implementation of the invention. If in step 376 it is determined that the maximum number of repetitions of the method has not been reached, the method returns to step 373.

If it is determined that the charge measurement stop condition has been reached, for example by the charge measurement has falling below a predetermined threshold, in step 375, thereby indicating that the vessel has been sealed, the method progresses to step 379 where a final set of microwave pulses are provided to the tissue in order to ensure a true seal before stopping the delivery of microwaves in step 380. An audible alert indicating the completion of the method is sounded in step 281 and the method finished in step 382.

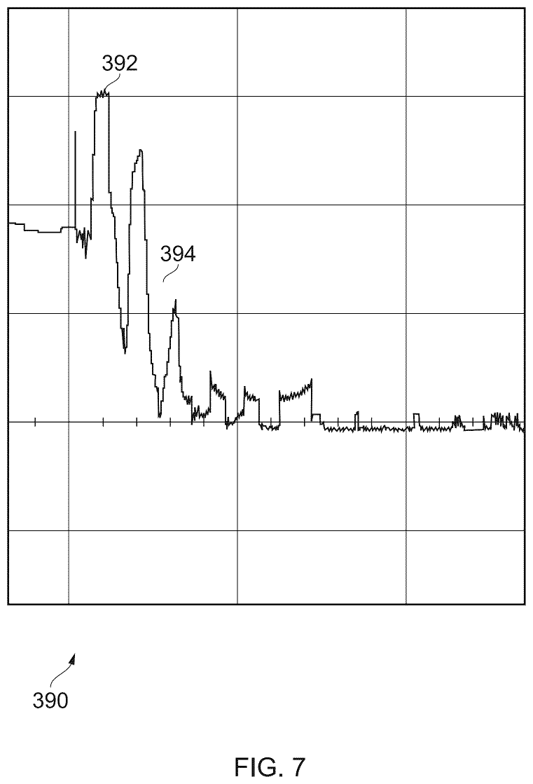

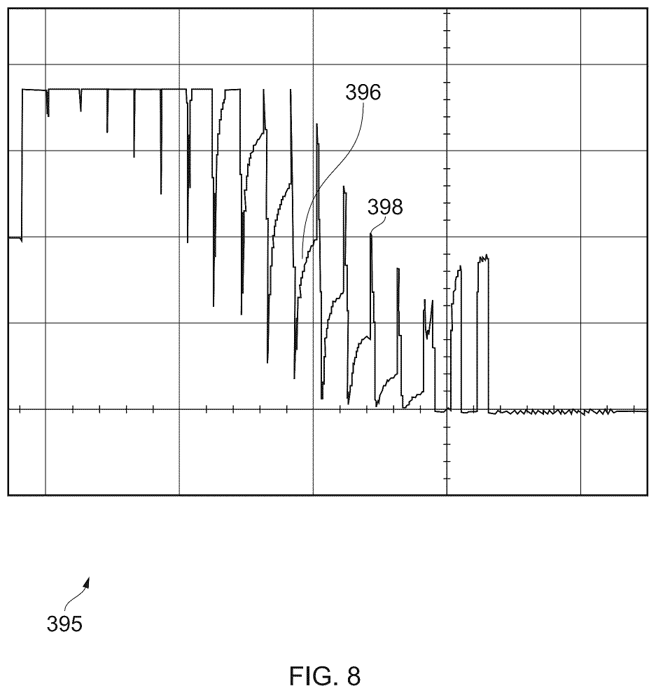

FIGS. 7 and 8 show plots 390, 395 illustrating the charge measurement for a vessel undergoing a microwave based sealing treatment. These plots are representative of charge measurements obtained using a control signal at 500 kHz and it should be noted that the observed behaviour of the charge measurements may be different at different frequencies. In both plots, the peaks 392, 396 represent the charge measurements obtained during each measurement window and the troughs 394, 398 represent the treatment windows where the microwave radiation is being applied to the vessel, meaning that the charge measurement apparatus is deactivated as described above. As illustrated in both plots, the charge measurement approaches zero as more microwave radiation is applied to the vessel. In the examples illustrated in FIGS. 7 and 8, the vessels are sealed when the charge measurements are substantially zero.

FIG. 9 shows a schematic diagram of an electrosurgical apparatus 400 such as that disclosed in GB 2 486 343 that is useful for understanding the invention. The apparatus comprises a RF channel and a microwave channel. The RF channel contains components for generating and controlling an RF frequency electromagnetic signal at a power level suitable for treating (e.g., cutting or desiccating) biological tissue. The microwave channel contains components for generating and controlling a microwave frequency electromagnetic signal at a power level suitable for treating (e.g., coagulating or ablating) biological tissue.

The microwave channel has a microwave frequency source 402 followed by a power splitter 424 (e.g., a 3 dB power splitter), which divides the signal from the source 402 into two branches. One branch from the power splitter 424 forms a microwave channel, which has a power control module comprising a variable attenuator 404 controlled by controller 406 via control signal V10 and a signal modulator 408 controlled by controller 406 via control signal V11, and an amplifier module comprising drive amplifier 410 and power amplifier 412 for generating forward microwave EM radiation for delivery from a probe 420 at a power level suitable for treatment. After the amplifier module, the microwave channel continues with a microwave signal coupling module (which forms part of a microwave signal detector) comprising a circulator 416 connected to deliver microwave EM energy from the source to the probe along a path between its first and second ports, a forward coupler 414 at the first port of the circulator 416, and a reflected coupler 418 at the third port of the circulator 416. After passing through the reflected coupler, the microwave EM energy from the third port is absorbed in a power dump load 422. The microwave signal coupling module also includes a switch 415 operated by the controller 406 via control signal V12 for connecting either the forward coupled signal or the reflected coupled signal to a heterodyne receiver for detection.

The other branch from the power splitter 424 forms a measurement channel. The measurement channel bypasses the amplifying line-up on the microwave channel, and hence is arranged to deliver a low power signal from the probe. A primary channel selection switch 426 controlled by the controller 406 via control signal V13 is operable to select a signal from either the microwave channel or the measurement channel to deliver to the probe. A high band pass filter 427 is connected between the primary channel selection switch 426 and the probe 420 to protect the microwave signal generator from low frequency RF signals.

The measurement channel includes components arranged to detect the phase and magnitude of power reflected from the probe, which may yield information about the material e.g., biological tissue present at the distal end of the probe. The measurement channel comprises a circulator 428 connected to deliver microwave EM energy from the source 402 to the probe along a path between its first and second ports. A reflected signal returned from the probe is directed into the third port of the circulator 428. The circulator 428 is used to provide isolation between the forward signal and the reflected signal to facilitate accurate measurement. However, as the circulator does not provide complete isolation between its first and third ports, i.e., some of the forward signal may break through to the third port and interfere with the reflected signal, a carrier cancellation circuit may be used that injects a portion of the forward signal (from forward coupler 430) back into the signal coming out of the third port (via injection coupler 432). The carrier cancellation circuit include a phase adjustor 434 to ensure that the injected portion is 180° out of phase with any signal that breaks through into the third port from the first port in order to cancel it out. The carrier cancellation circuit also include a signal attenuator 436 to ensure that the magnitude of the injected portion is the same as any breakthrough signal.

To compensate for any drift in the forward signal, a forward coupler 438 is provided on the measurement channel. The coupled output of the forward coupler 438 and the reflected signal from the third port of the circulator 428 are connected to respective input terminal of a switch 440, which is operated by the controller 406 via control signal V14 to connect either the coupled forward signal or the reflected signal to a heterodyne receiver for detection.

The output of the switch 440 (i.e. the output from the measurement channel) and the output of the switch 415 (i.e. the output from the microwave channel) are connect to a respective input terminal of a secondary channel selection switch 442, which is operable by the controller 406 via control signal V15 in conjunction with the primary channel selection switch to ensure that the output of the measurement channel is connected to the heterodyne receiver when the measurement channel is supplying energy to the probe and that the output of the microwave channel is connected to the heterodyne receiver when the microwave channel is supplying energy to the probe.

The heterodyne receiver is used to extract the phase and magnitude information from the signal output by the secondary channel selection switch 442. A single heterodyne receiver is shown in this system, but a double heterodyne receiver (containing two local oscillators and mixers) to mix the source frequency down twice before the signal enters the controller may be used if necessary. The heterodyne receiver comprises a local oscillator 444 and a mixer 448 for mixing down the signal output by the secondary channel selection switch 442. The frequency of the local oscillator signal is selected so that the output from the mixer 448 is at an intermediate frequency suitable to be received in the controller 406. Band pass filters 446, 450 are provided to protect the local oscillator 444 and the controller 406 from the high frequency microwave signals.

The controller 406 receives the output of the heterodyne receiver and determines (e.g., extracts) from it information indicative of phase and magnitude of the forward and/or reflected signals on the microwave or measurement channel. This information can be used to control the delivery of high power microwave EM radiation on the microwave channel or high power RF EM energy on the RF channel. A user may interact with the controller 406 via a user interface 452.

The RF channel shown in FIG. 9 comprises an RF frequency source 454 connected to a gate driver 456 that is controlled by the controller 406 via control signal V16. The gate driver 456 supplies an operation signal for an RF amplifier 458, which is a half-bridge arrangement. The drain voltage of the half-bridge arrangement is controllable via a variable DC supply 460. An output transformer 462 transfers the generated RF signal on to a line for delivery to the probe 420. A low pass, band pass, band stop or notch filter 464 is connected on that line to protect the RF signal generator from high frequency microwave signals.

A current transformer 466 is connected on the RF channel to measure the current delivered to the tissue load. A potential divider 468 (which may be tapped off the output transformer) is used to measure the voltage. The output signals from the potential divider 468 and current transformer 466 (i.e., voltage outputs indicative of voltage and current) are connected directly to the controller 406 after conditioning by respective buffer amplifiers 470, 472 and voltage clamping Zener diodes 474, 476, 478, 480 (shown as signals B and C in FIG. 9).

To derive phase information, the voltage and current signals (B and C) are also connected to a phase comparator 482 (e.g., an XOR gate) whose output voltage is integrated by RC circuit 484 to produce a voltage output (shown as A in FIG. 9) that is proportional to the phase difference between the voltage and current waveforms. This voltage output (signal A) is connected directly to the controller 406.

The microwave/measurement channel and RF channel are connected to a signal combiner 417, which conveys both types of signal separately or simultaneously along cable assembly 419 to the probe 420, from which it is delivered (e.g., radiated) into the biological tissue of a patient.

A waveguide isolator (not shown) may be provided at the junction between the microwave channel and signal combiner 417. The waveguide isolator may be configured to perform three functions: (i) permit the passage of very high microwave power (e.g., greater than 10 W); (ii) block the passage of RF power; and (iii) provide a high withstanding voltage (e.g., greater than 10 kV). A capacitive structure (also known as a DC break) may also be provided at (e.g., within) or adjacent the waveguide isolator. The purpose of the capacitive structure is to reduce capacitive coupling across the isolation barrier.

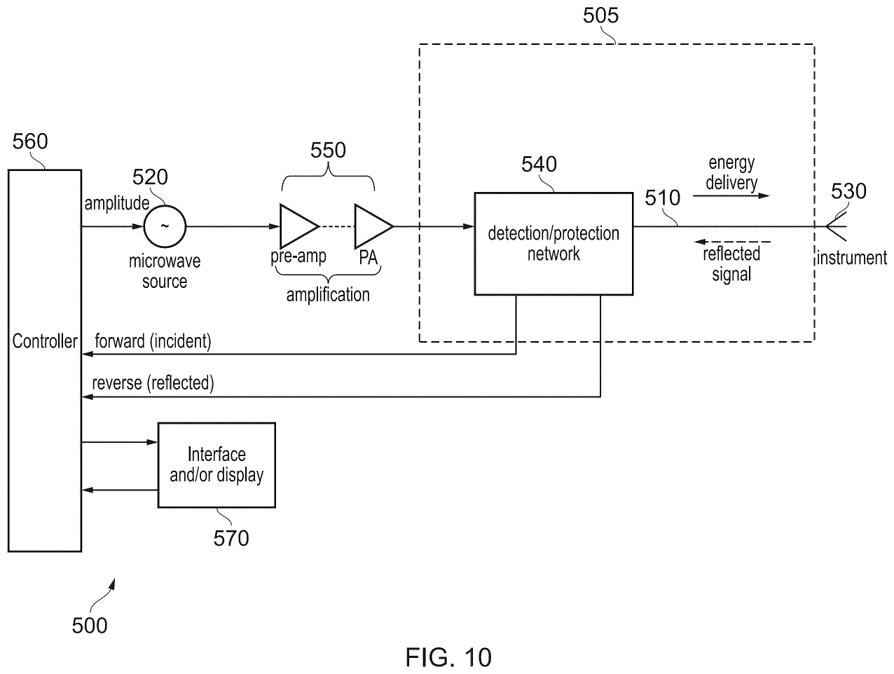

FIG. 10 shows an electrosurgical system 500 including an apparatus 505 for sensing a condition of a biological tissue. The apparatus comprises a feed structure 510, such as a coaxial cable, comprising a microwave channel for connecting a microwave signal generator 520 to a probe 530 for delivering microwave radiation into the biological tissue. The apparatus further comprises a microwave signal detector, which is part of a detection and protection circuit 540, adapted to: sample reflected power on the microwave channel in response to conveying the microwave radiation to the biological tissue; and generate, from the sampled reflected power, a microwave detection signal indicative of the microwave power reflected by the tissue. The delivery of microwave radiation to a biological tissue will cause the structure of the biological tissue to change, which will in turn change the impedance of the biological tissue and so will change the relationship between the impedance of the probe and the impedance of the tissue. As the reflected microwave power is indicative of this relationship, i.e., the impedance mismatch between probe and tissue, the microwave detection signal obtained from the sampled reflected power is indicative of the condition of the biological tissue.

The microwave detection signal comprises an amplitude of the sampled reflected power and/or a phase of the sampled reflected power. The microwave signal detector is further adapted to sample forward power on the microwave channel in response to conveying the microwave radiation to the biological tissue and generate, the microwave detection signal based on both the sampled forward power and the sampled reflected power. Accordingly, the microwave signal detector may be arranged to obtain microwave signal information that is indicative of the magnitude and/or phase of both the microwave EM radiation provided to, and reflected by, the biological tissue. The microwave detection signal may therefore also comprise a ratio of the amplitude of the sampled forward power to the amplitude of the sampled reflected power and/or a phase shift between the sampled forward power and the sampled reflected power.

The system 500 comprises a microwave signal generator 520 adapted to generate the microwave radiation to be delivered to the tissue. The microwave signal generator may comprise any microwave frequency source. The microwave signal generator may comprise an amplification unit 550 adapted to receive a pre-amplified microwave signal from the microwave frequency source and generate the microwave radiation to be provided to the biological tissue by amplifying the pre-amplified microwave signal.

The system 500 further comprises a probe 530 having a distal tip portion for delivering the microwave radiation into the biological tissue and for obtaining the microwave power reflected by the biological tissue, wherein the probe is in communication with the feed structure. The probe may comprise an antenna to radiate the microwave radiation. The antenna may comprise a pair of electrodes provided at the distal tip portion of the probe, such as an inner conductor and an outer conductor of a coaxial cable. The electrosurgical system 500 further comprises a processor 560 in communication with the apparatus 505. The processor is adapted to receive the microwave detection signal from the apparatus and determine a condition of the biological tissue based on the received microwave detection signal. For example, the processor may be adapted to determine if the tissue has been treated based on the microwave detection signal. The processor may be adapted to determine the condition of the biological tissue based on the microwave detection signal using a look-up table, a user defined condition, a neural network, and the like. The processor may be adapted to determine a complex impedance of the biological tissue based on a sampled forward power and a sampled reflected power in response to conveying the microwave radiation to the biological tissue.

As discussed above, the biological tissue may be a vessel and the treatment being applied may be sealing the vessel using the microwave radiation, in which case the processor 560 is adapted to determine when the vessel has been sealed based on the microwave detection signal. For example, a change in the microwave detection signal may be used to determine that the vessel has been sealed. In a further example, the measurement of the microwave detection signal being greater than or less than a predetermined threshold may be used to determine that the vessel has been sealed. Further, a rate of change of the microwave detection signal crossing a predetermined value may also be used to determine that the vessel has been sealed. Further, the rate of change of the microwave detection signal approaching a steady state may also be used to determine that the vessel has been sealed.

The biological tissue may also be any other type of tissue undergoing treatment, and in particular undergoing treatment involving the delivery of energy to the biological tissue. The delivery of energy to a biological tissue causes the structure of the biological tissue to change, which in turn changes the impedance mismatch between the probe and the biological tissue. For example, the biological tissue may be a tissue being resected, desiccated, ablated and the like. For example, the biological tissue may be a tumour undergoing ablation.

The processor 560 is further adapted to automatically deactivate the microwave signal generator 520 based on the determined condition of the biological tissue. In particular, the processor may be adapted to automatically deactivate the microwave signal generator if the vessel is determined as being sealed. In this way, excessive application of microwave radiation to a biological tissue may be avoided.

Further, the processor may be adapted to generate an alarm signal if the vessel is not determined as being sealed within a predetermined time period. The predetermined time period may be 5 seconds. The system may comprise a user interface 570 adapted to receive the alarm signal and generate an alert to be provided to a user of the system, such as a clinician. The user interface may comprise one or more of: an audio output, wherein the alarm signal is adapted to cause the audio output to generate an audio signal as the alert; a visual output, wherein the alarm signal is adapted to cause the visual output to generate an visual signal as the alert; an haptic output, wherein the alarm signal is adapted to cause the haptic output to generate an haptic signal as the alert; and the like.

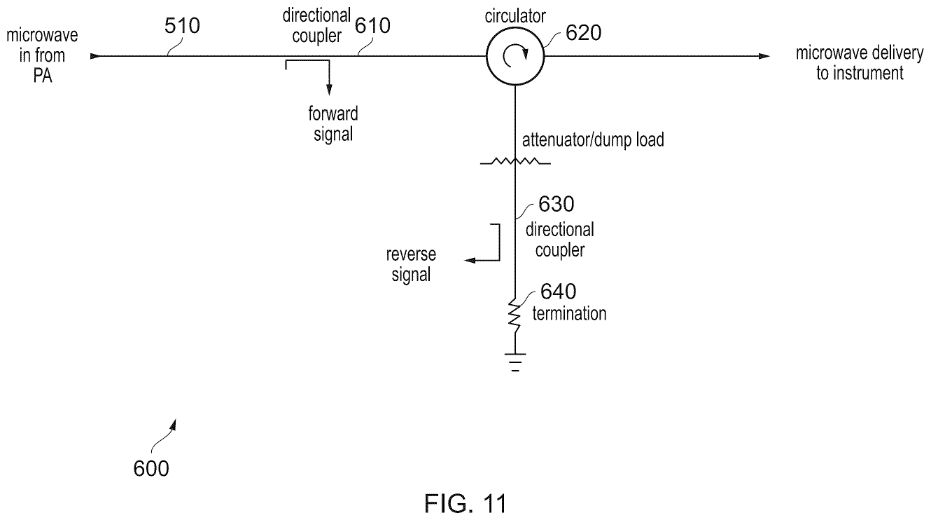

FIGS. 11 to 14 show several embodiments of the detection and protection circuit 540 in FIG. 10.

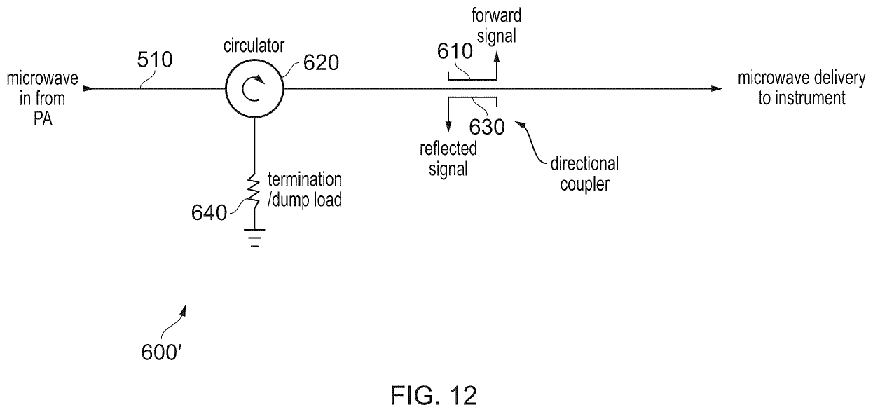

In FIG. 11, the detection and protection circuit 600 comprises a first directional coupler 610 in communication with the microwave channel of the feed structure 510 and upstream of a circulator 620. The first directional coupler samples the forward power on the microwave channel. The circuit further comprises a second directional coupler 630 in communication with the microwave channel of the feed structure 510 and downstream of a circulator 620. In use, the microwave radiation to be delivered to the biological tissue travels towards the circulator and is sampled by the first directional coupler. The circulator then directs the microwave radiation towards the biological tissue. Reflected microwave radiation travels back through the feed structure towards the circulator, which directs the reflected microwave radiation to a termination load 640. Between the circulator and the termination load, the reflected microwave radiation is sampled by the second directional coupler. The detection and protection circuit 600′in FIG. 12 is identical to the one shown in FIG. 11, except that both direction couplers are provided downstream of the circulator 620 between the circulator and the biological tissue.

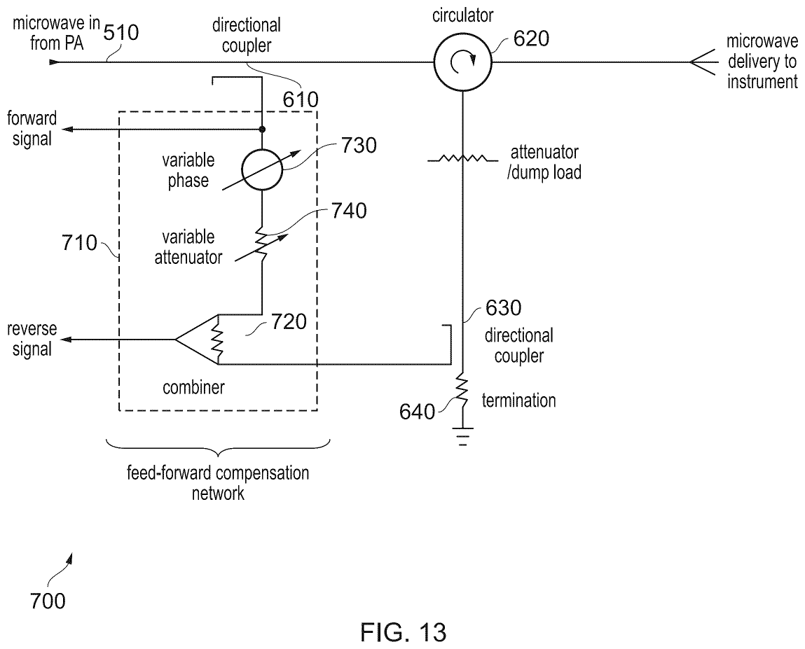

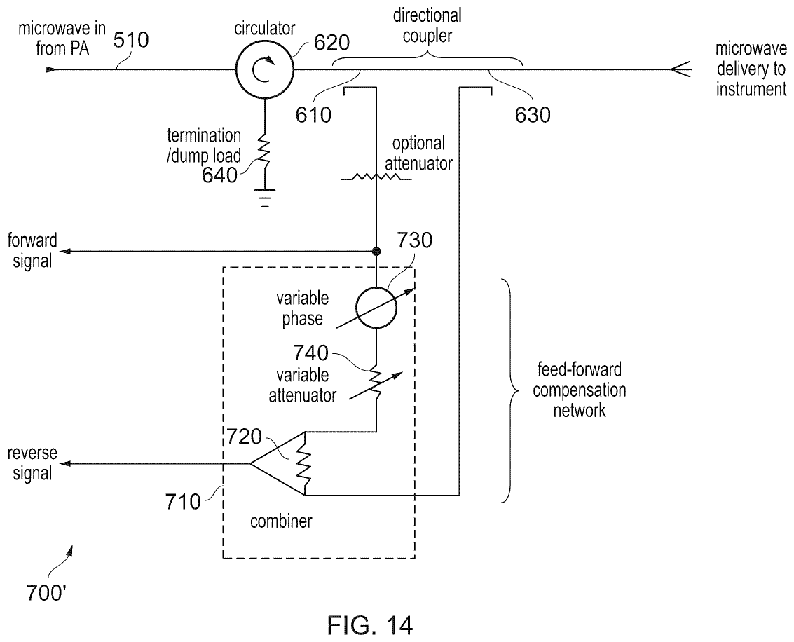

In the detection and protection circuit 700 shown in FIG. 13 the first directional coupler 610 is upstream of the circulator 620. The second directional coupler 630 is downstream of the circulator 620 and between the circulator and the termination load 640. The circuit 700 further comprises a compensation unit 710 adapted to correct the microwave detection signal. The compensation unit comprises a combiner 720 for combining the sampled reflected power and the sampled forward power. The compensation unit further comprises a phase shifter 730 and a variable attenuator 740. In use, the microwave radiation to be delivered to the biological tissue travels towards the circulator and is sampled by the first directional coupler. The circulator then directs the microwave radiation towards the biological tissue. Reflected microwave radiation travels back through the feed structure towards the circulator, which directs the reflected microwave radiation to the termination load 640. Between the circulator and the termination load, the reflected microwave radiation is sampled by the second directional coupler. The forward power signal obtained by the first directional coupler is both sent for use in determining the microwave detection signal and provided to the compensation unit. The forward power signal received by the compensation unit has a phase shift imparted on it by the phaser shifter and is attenuated by the variable attenuator before being combined with the reflected power signal obtained by the second directional coupler. The detection and protection circuit 700′ in FIG. 14 is identical to the one shown in FIG. 13, except that both direction couplers are provided downstream of the circulator 620 between the circulator and the biological tissue. The compensation unit 710 is compensating for the non-ideal directivity of the directional couplers, i.e., the difference between coupling and isolation factors. Unwanted measurement of the forward which couples to the reverse measurement, or vice-versa.

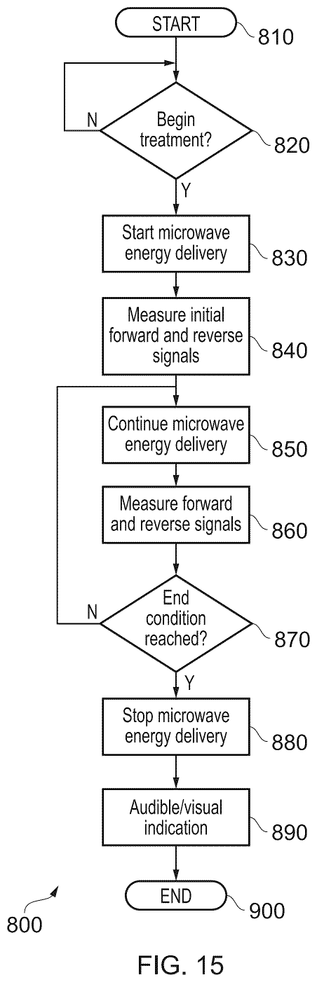

FIG. 15 shows a flow diagram 800 illustrating a method for using the system shown in FIG. 10.

The method begins in step 810 and proceeds to step 820 to determine whether the system has been activated for delivering microwave energy to a biological tissue. The system waits until the trigger signal is received and then progresses to step 830, where microwave radiation is applied to the biological tissue. The initial reflected microwave radiation is measured in step 840, which can give an idea of the initial impedance mismatch between the probe and the biological tissue.

Microwave radiation is then continually applied in step 850 and the microwave detection signals obtained in step 860 in order to determine in step 870 whether an end condition has been reached, as described above. If the end condition has not been reached the method returns to step 850. If the end condition has been reached the method proceeds to step 880 where the delivery of microwave energy stops, an alert is provided to the user in step 890 and the method finishes in step 900.

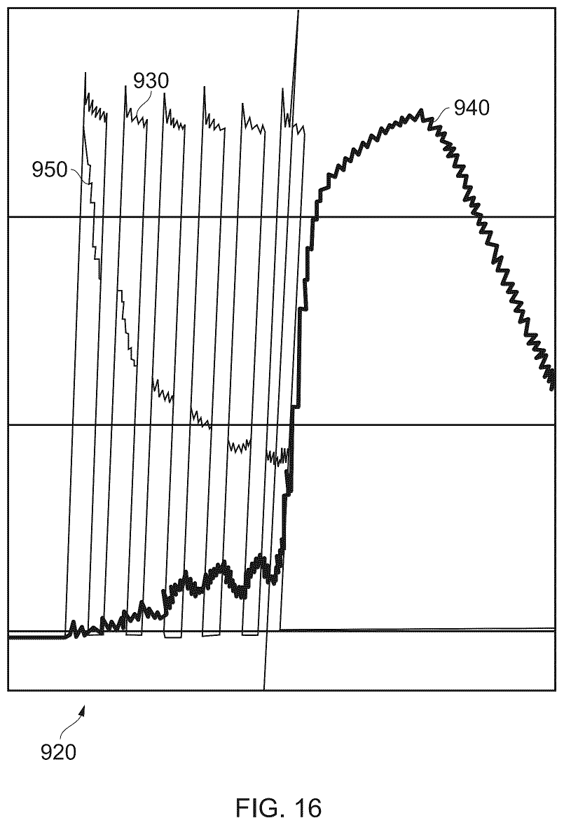

FIG. 16 shows a plot 920 showing the application of microwave radiation 930 to the biological tissue, the charge measurement 940 obtained as described above and the microwave detection signal 950 obtained as described above. As can be seen in FIG. 16, the charge measurement 940 rapidly increases as the microwave detection signal 950 approaches a steady state, both of which may be indicative of the completion of the sealing of a vessel by the application of the microwave radiation.

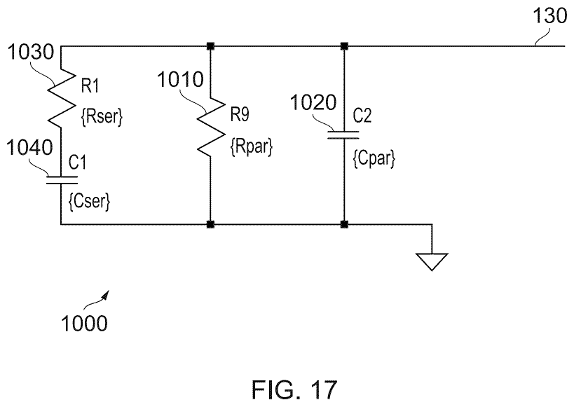

FIG. 17 shows a schematic representation of a lumped element model 1000 used to model biological tissue and instrument frequency responses.

The lumped element model comprises Rpar 1010, Cpar 1020, Rser 1030 and Cser 1040. Cpar mostly comprises the capacitance of the cable between the sensing circuit and the instrument. Rpar models conductive contamination, as well as the DC resistance of the tissue. Rser and Cser represent the relaxation time constant of the tissue. The intent of the model 1000 is to measure the frequency response due to Rser and Cser, as this contains information about the tissue type and state/condition of the tissue, whilst rejecting any effect due to Cpar or Rpar, i.e., rejecting the effects of cable capacitance and conductive contamination.

Typical measurements of tissue resistance, which essentially measure Rpar, are vulnerable to being shorted out by conductive contamination. Typical impedance measurements would include the AC current flowing through Cpar. At typical electrosurgical frequencies of 400 kHz, and for cable lengths of several metres as typically used in endoscopic and robotic surgery, Cpar can dominate the impedance measurement, making the measurement of impedance changes due to the tissue challenging.

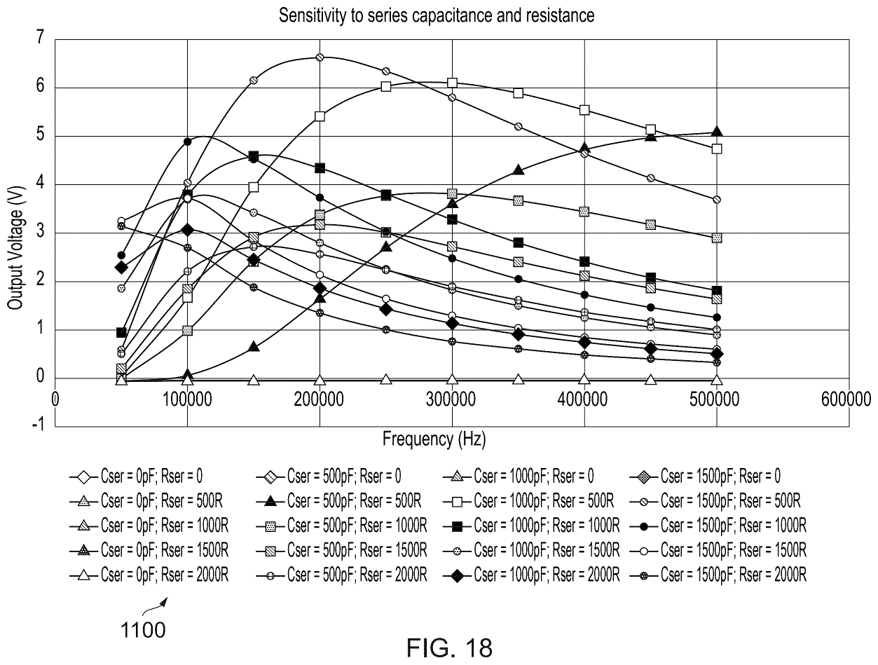

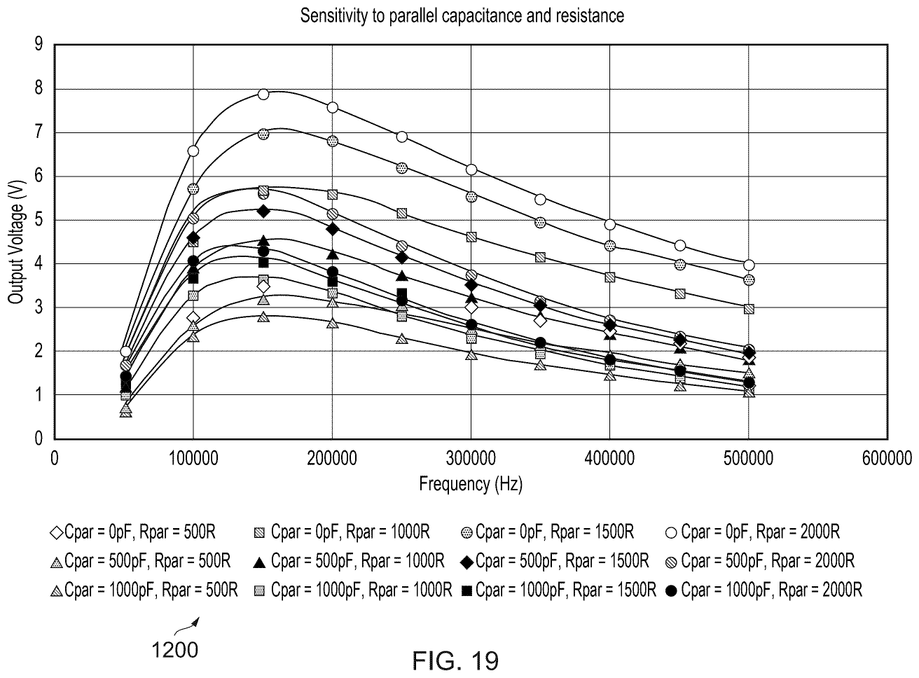

FIGS. 18 and 19 illustrate the simulated frequency response of a lumped element model of the tissue (FIG. 18) and system components (FIG. 19) as the resistance and capacitance of the lumped element model change.

As the storage of positive and negative charges is approximately equal in the tissue, and the charge storage behaves linearly, it is possible to simulate the behaviour of the circuit using a linear lumped element model containing resistors and capacitors.

The lumped element model comprises Rpar, Cpar, Rser and Cser. In practice, Cpar was found to mostly comprise of the capacitance of the cable between the sensing circuit and the instrument. Rpar models conductive contamination, as well as the DC resistance of the tissue. Rser and Cser represent the relaxation time constant of the tissue. The intent of the simulation is to measure the frequency response due to Rser and Cser, as this contains information about the tissue type and state of the tissue, whilst ideally rejecting any effect due to Cpar or Rpar, i.e., rejecting the effects of cable capacitance and conductive contamination.

This differs from the measurement of tissue resistance, which essentially measures Rpar and therefore is vulnerable to being shorted out by conductive contamination, or impedance measurement which also includes the AC current flowing through Cpar. At typical electrosurgical frequencies of 400 kHz, and for cable lengths of several metres as typically used in endoscopic and robotic surgery, Cpar can dominate the impedance measurement, making the measurement of impedance changes due to the tissue challenging.

Each simulation step clocks the analogue switch at the defined frequency, runs until steady state is achieved and takes a measurement of the average output voltage. Parameterising the lumped element model allows the sensitivity of the output voltage to the value each element to be determined, which illustrates how the measurement circuit is able to extract tissue information.

FIG. 18 shows a graph 1100 of amplitude of output voltage in V (Y-axis) against frequency in Hz (X-axis) to illustrate how the shape of the frequency response and the peak frequency varies with the value of Cser and Rser, which represent the electrical model of the dielectric relaxation within the tissue. The exact capacitance and resistance are not so important as the product of the two, which determines the time constant of the tissue and relates to the frequency at which the peak measurement is obtained. For example, doubling the size of the electrodes will halve the resistance but double the capacitance, with the time constant remaining the same.

As shown in FIG. 18, as the values of Cser and Rser change, so does the peak frequency. For example, if the treatment involves applying heat to tissue, generally the product of resistance and capacitance decreases, meaning that the peak frequency decreases as heat is applied. One example of this is vessel sealing, in which the change occurs rapidly around the moment of optimal seal, but this has also found to be true of heating liver and connective tissue in which the change is more gradual.

It is notable that the traces along the x-axis represent an open circuit, and the response remains at zero throughout the whole frequency range, illustrating the fact that there is no signal contribution from the elements Cpar and Rpar due to the intermediate discharging state between application of charge and measurement. The shape of the measured frequency response is thus dependent upon the dielectric relaxation properties of the measured tissue, as modelled by lumped elements Cser and Rser, and not a function of the cable capacitance or conductive contamination.

FIG. 19 shows a graph 1200 of amplitude against frequency to illustrate how the shape of the frequency response remains unchanged as Rpar and Cpar are swept over a range of realistic values. The effect of adding parallel resistance or capacitance is to vary the amplitude of the frequency response, but not the shape of the response. Thus, by comparing the measured signal at two frequencies measured in rapid succession as the trigger to cut off energy delivery, or by using some other method of analysing the shape of the normalised frequency response, this trigger point is made insensitive to conductive contamination and cable capacitance.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An apparatus for sensing a condition or type of a biological tissue, the apparatus comprising:
a control circuit, wherein the control circuit comprises an electrical connection for delivering electrical signals to the biological tissue, and wherein the control circuit is adapted to:
deliver an electrical signal to the biological tissue for an active time period; and
obtain a charge measurement of an amount of charge stored in the biological tissue in response to the electrical signal during a sampling period after the active time period.

2. The apparatus as claimed in claim 1, wherein the control circuit comprises a switching unit adapted to switch the control circuit between a plurality of operation modes, the plurality of operation modes comprising:
an active mode, wherein the electrical connection is connected to an electrical signal source during the active time period; and
a floating mode, wherein the electrical connection is connected to a sensing ground during the sampling period, thereby obtaining the charge measurement.

3. The apparatus according to claim 2, wherein the plurality of operation modes further comprises a reference ground mode, wherein the electrical connection is connected to a reference ground during the reference ground mode.

4. The apparatus according to claim 3, wherein the switching unit is adapted to switch the control circuit to the reference ground mode after the active time period and before the sampling period.

5. The apparatus according to claim 2, wherein the plurality of operation modes further comprises an error compensating mode, wherein the electrical connection is connected to an offset signal source during the error compensating mode.

6. The apparatus according to claim 5, wherein the switching unit is adapted to switch the control circuit to the error compensating mode after the active time period and before the sampling period.

7. The apparatus according to claim 3, wherein the switching unit alternates between selecting the reference ground mode and an error compensating mode between subsequent active modes.

8. The apparatus according to claim 2, wherein the active mode comprises:
a first active mode, wherein the electrical connection is connected to an electrical signal source having a first polarity; and
a second active mode, wherein the electrical connection is connected to an electrical signal source having a second polarity, opposite the first polarity;
wherein the switching unit is adapted to select one of the first active mode and the second active mode during the active mode.

9. The apparatus according to claim 8, wherein the switching unit alternates between selecting the first active mode and selecting the second active mode between subsequent active modes.

10. The apparatus according to claim 1, wherein the control circuit further comprises an amplifier unit adapted to amplify the charge measurement.

11. The apparatus according to claim 9, wherein the control circuit further comprises an amplifier unit comprising:
a first amplifier circuit adapted to amplify the charge measurement obtained in the floating mode following the first active mode; and
a second amplifier circuit adapted to amplify the charge measurement obtained in the floating mode following the second active mode.

12. The apparatus according to claim 11, wherein the amplifier unit further comprises a third amplifier circuit adapted to obtain a difference between the amplified charge measurement from the first amplifier circuit and the amplified charge measurement from the second amplifier circuit.

13. The apparatus according to claim 2, wherein the control circuit comprises an input for receiving a control signal, and wherein the active time period is based on the control signal.

14. The apparatus according to claim 13, wherein the apparatus further comprises signal generator in communication with the input, wherein the signal generator is adapted to generate the control signal.

15. The apparatus according to claim 13, wherein the control circuit comprises a counter adapted to generate a clocking signal based on the control signal, and wherein the switching unit is adapted to cycle the control circuit through the plurality of operation modes based on the clocking signal.

16. The apparatus according to claim 13, wherein the control signal has a frequency between 100 Hz and 100 MHz.

17. The apparatus according to claim 13, wherein the control signal is swept between a plurality of frequencies.

18. An electrosurgical system for treating a biological tissue, the electrosurgical system comprising:
the apparatus according to claim 1 and
a probe having a distal tip portion for delivering the electrical signal into the biological tissue and for obtaining the charge measurement from the tissue, wherein the probe is in electrical communication with the electrical connection.

19. The electrosurgical system according to claim 18, wherein the electrosurgical system further comprises a processor in communication with the apparatus, wherein the processor is adapted to:
receive the charge measurement from the apparatus; and
determine a condition or type of the biological tissue based on the received charge measurement.

20. The electrosurgical system according to claim 19, wherein the electrosurgical system further comprises a microwave signal generator for generating microwave electromagnetic radiation, wherein the probe is further adapted to deliver the microwave electromagnetic radiation.

21. The electrosurgical system according to claim 20, wherein the processor is further adapted to:
activate the apparatus for obtaining the charge measurement from the biological tissue and deactivate the microwave signal generator during a charge measurement window, which has a charge measurement time period; and
deactivate the apparatus for obtaining the charge measurement from the biological tissue and activate the microwave signal generator during a treatment window, which has a treatment time period.

22. The electrosurgical system according to claim 20, wherein the processor is further adapted to automatically deactivate the microwave signal generator based on the determined condition of the biological tissue.

23. The electrosurgical system according to claim 22, wherein the biological tissue is a vessel, and wherein the processor is adapted to automatically deactivate the microwave signal generator if the vessel is determined as being sealed based on one or more of:
the charge measurement crossing a predetermined threshold;
a rate of change of the charge measurement;
a rate of change of the charge measurement approaching a steady state; and
a rate of change of the charge measurement crossing a predetermined threshold.

24. The electrosurgical system according to claim 23, wherein the processor is adapted to generate an alarm signal if the vessel is not determined as being sealed within a predetermined time period.

25. The electrosurgical system according to claim 20, wherein the processor is further adapted to automatically deactivate the microwave signal generator based on the determined type of the biological tissue.

26. The electrosurgical system according to claim 20, wherein the processor is adapted to generate an alert signal based on the determined type of the biological tissue.

27. An electrosurgical apparatus for sensing a condition or type of a biological tissue, the apparatus comprising:
a control circuit, wherein the control circuit comprises an electrical connection for delivering electrical signals to the biological tissue, and wherein the control circuit is adapted to:
deliver an electrical signal to the biological tissue for an active time period;
obtain a charge measurement of an amount of charge stored in the biological tissue in response to the electrical signal during a sampling period after the active time period;
determine a tissue type of the biological tissue based on the charge measurement; and
generate a control signal for delivery to an energy generator for controlling the delivery of energy to the biological tissue based on the determined tissue type.

28. The electrosurgical apparatus according to claim 27, wherein the delivering the electrical signal comprises delivering an electrical signal at a plurality of signal frequencies.

29. The electrosurgical apparatus according to claim 28, wherein obtaining the charge measurement comprises obtaining a charge measurement at each of the plurality of signal frequencies, thereby obtaining a plurality of charge measurements.

30. The electrosurgical apparatus according to claim 29, wherein determining the tissue type of the biological tissue is based on the plurality of charge measurements.

31. The electrosurgical apparatus according to claim 30, wherein determining the tissue type of the biological tissue based on the plurality of charge measurements comprises one or more of:
determining the tissue type based on a signal frequency associated with a peak charge measurement from the plurality of charge measurements; and
determining the tissue type based on a signal frequency associated with a minimum charge measurement from the plurality of charge measurements.

32. The electrosurgical apparatus according to claim 27, wherein, if the determined tissue type is muscle, generating the control signal comprises generating a stop signal for stopping the delivery of energy to the biological tissue.

33. The electrosurgical apparatus according to claim 32, wherein, if the tissue type is one or more of mucosa, sub-mucosa and epithelium, generating the control signal comprises generating a delivery signal for starting the delivery of energy to the biological tissue.

34-48. (canceled)

49. A system for monitoring a biological tissue, the system comprising:
the electrosurgical apparatus for sensing a condition or type of biological according to claim 1; and
an electrosurgical apparatus for sensing a condition of a biological tissue, the apparatus comprising:
a feed structure comprising a microwave channel for connecting a microwave signal generator to a probe for delivering microwave radiation into the biological tissue; and
a microwave signal detector adapted to:
sample reflected power on the microwave channel in response to conveying the microwave radiation to the biological tissue; and
generate, from the sampled reflected power, a microwave detection signal indicative of the microwave power reflected by the tissue

50. The system according to claim 49 further comprising the electrosurgical apparatus for sensing a condition or type of a biological tissue, the apparatus comprising:
a control circuit, wherein the control circuit comprises an electrical connection for delivering electrical signals to the biological tissue, and wherein the control circuit is adapted to:
deliver an electrical signal to the biological tissue for an active time period;
obtain a charge measurement of an amount of charge stored in the biological tissue in response to the electrical signal during a sampling period after the active time period;
determine a tissue type of the biological tissue based on the charge measurement; and
generate a control signal for delivery to an energy generator for controlling the delivery of energy to the biological tissue based on the determined tissue type.
